(19) United States
(12) Patent Application Publication
(10) Pub. No.: US 2024/0189433 A1
(43) Pub. Date: Jun. 13, 2024

(54) SUPERABSORBENT HYDROGELS

(71) Applicant: JUNION LABS PTE. LTD., Singapore (SG)

(72) Inventors: Jingnan LUO, Singapore (SG); Hongqian BAO, Singapore (SG); Bo Huai Moses LEE, Singapore (SG)

(73) Assignee: JUNION LABS PTE. LTD., Singapore (SG)

(21) Appl. No.: 18/551,592

(22) PCT Filed: Mar. 23, 2022
(86) PCT No.: PCT/SG2022/050161

§ 371 Date: Sep. 20, 2023

(30) Foreign Application Priority Data
Mar. 23, 2021 (SG) 10202102990S

(51) Int. Cl. A61K 47/60; A61K 9/00; A61K 9/06; A61P 3/04

(52) U.S. Cl. CPC A61K 47/60; A61K 9/0053; A61K 9/06; A61P 3/04

(57) ABSTRACT

The present invention relates to polymer comprising a polysaccharide crosslinked with a spacer crosslinker, wherein the spacer crosslinker comprises a first optionally substituted aliphatic moiety terminated at each end with a second moiety comprising at least two carboxylic acid groups. The present invention also relates to a hydrogel, a method of forming the polymer or hydrogel, a composition and capsule comprising the polymer or hydrogel and a method treating obesity, pre-diabetes, diabetes, non-alcoholic fatty liver disease or chronic idiopathic constipation, or of reducing caloric intake or improving glycemic control using the polymer or hydrogel and a method of weight-loss or improving the body appearance in a healthy subject.

### TECHNICAL FIELD

The present invention relates to a polymer comprising a polysaccharide crosslinked with a spacer crosslinker. The present invention also relates to a hydrogel, method of forming the polymer or hydrogel, a composition and capsule comprising the polymer or hydrogel and a method of treating obesity, pre-diabetes, diabetes, non-alcoholic fatty liver disease, chronic idiopathic constipation, reducing caloric intake or improving glycemic control using the polymer or hydrogel.

### BACKGROUND ART

A hydrogel is a network of crosslinked polymer chains that are hydrophilic and are able to absorb aqueous solutions through hydrogen bonding with water molecules. The water molecules are retained within the hydrogel leading to the hydrogel swelling to multiple times its original volume in the process. The structural integrity of the hydrogel network is maintained in water due to the crosslinking that holds the hydrophilic polymer chains together, forming a three dimensional solid. Superabsorbent polymer hydrogels (SAPs) are hydrogels which are able to absorb and retain extremely large amounts of a liquid relative to its own mass. In deionized and distilled water, a SAP may absorb 300 times its weigh (from 30 to 60 times its own volume) and can become up to 99.9% liquid.

The total absorbency and swelling capacity of a hydrogel is controlled by the type and degree of crosslinkers used to make the gel. For example, low-density crosslinked SAPs generally have a higher absorbent capacity and swell to a larger degree, resulting in a softer and stickier hydrogel formation. In contrast, SAPs with a high crosslinking density exhibits a lower absorbent capacity and swelling, but the gel strength is higher and its particle shape can be maintained even under modest pressure.

However, in either case, in known SAPs, the polymer chains are randomly crosslinked with little control over the structure of the resulting polymeric network, and therefore the properties of the polymer is not easily predictable. Further, conventional SAPs use short crosslinkers which result in compacted and less flexible polymer networks, which in turn results in decreased mechanical strength and water absorbance of the polymer and hydrogel. Further, conventional SAPs that are commercially available are almost exclusively acrylic-based products, which lack biodegradability.

There is therefore a need to provide a polymer or hydrogel which overcomes or at least ameliorates, one or more of the disadvantages described above.

### SUMMARY

In an aspect, there is provided a polymer comprising a polysaccharide crosslinked with a spacer crosslinker, wherein the spacer crosslinker comprises a first optionally substituted aliphatic moiety terminated at each end with a second moiety comprising at least two carboxylic acid groups.

In another aspect, there is provided a hydrogel comprising the polymer as defined as above and a liquid.

Advantageously, the polymer or hydrogel as defined above are crosslinked with a spacer crosslinker which form generally more stable and stiff networks compared to polymers or hydrogels that are associated by non-chemical, physical interactions only. Further, contrary to known polymers and hydrogels where the spacer group is randomly crosslinked with the polysaccharide, the polymer of the present application uses a pre-formed, well-defined spacer crosslinker. The structure, including the chain length and molecular weight of the spacer crosslinker, is therefore well-defined prior to crosslinking with the polysaccharide, hence the resulting polymer is more predictably formed and easily characterized. Advantageously, the polymer and hydrogel as defined above facilitates a more controlled modulation of the water absorbance of the polymer and hydrogel, which results in better performance in terms of mechanical strength and media uptake ratio compared to previously known hydrogels that are randomly crosslinked.

In another example, the first optionally substituted aliphatic molecule has a molecular weight in the range of about 0.1 kDa to about 100 kDa.

Advantageously, by using a long spacer crosslinker, crosslinking between the polysaccharide chains can occur in a more versatile and flexible manner, resulting in a polymer with a looser polymeric network while still having a high level of strength. A looser polymeric network further advantageously facilitates a greater swelling ratio of the hydrogel as it allows the polysaccharide chains within the network to move further away from each other, allowing for the polymer network to swell to a greater extent.

In another aspect, there is provided a method of forming the polymer as defined above, comprising the steps of:
a) reacting a first optionally substituted aliphatic molecule comprising at least two hydroxyl groups with a second molecule comprising at least three carboxylic acid groups to form a spacer crosslinker; and
b) crosslinking the spacer crosslinker with a polysaccharide to form the polymer of as defined above.

Advantageously, the method allows for the formation of the spacer crosslinker as well as the polymer with or without using a catalyst. Advantageously, the reaction may proceed in the absence of a catalyst, which eliminates the issue of lower yield due to the presence of residual catalyst or the need to remove residual catalyst. Further advantageously, since the structure of the crosslinker is known, it is possible to have better control over the amount of crosslinker to be used in the reaction.

More advantageously due to the long length of the spacer crosslinker, it is unlikely for crosslinking to occur within the same polysaccharide chain or for crosslinking to occur multiple times between the same two polysaccharide chains. Accordingly, the amount of crosslinker used in the reaction can be reduced, while still retaining a strong polymeric network structure, which is an economical advantage.

In another aspect, there is provided a composition comprising the polymer as defined above or the hydrogel as defined above and a pharmaceutically acceptable excipient.

In another aspect, there is provided a capsule comprising the polymer as defined above or the hydrogel as defined above.

In another aspect, there is provided a method of treating obesity, pre-diabetes, diabetes, non-alcoholic fatty liver disease (NAFLD), or chronic idiopathic constipation, or of reducing caloric intake or improving glycemic control in a subject in need thereof, comprising the step of orally administering to the subject a therapeutically effective amount of the polymer as defined above or the hydrogel as defined above or the composition as defined above.

In another aspect, there is provided the polymer as defined above or the hydrogel as defined above or the composition as defined above for use in the treatment of obesity, pre-diabetes, diabetes, non-alcoholic fatty liver disease (NAFLD), or chronic idiopathic constipation, or for reducing caloric intake or improving glycemic control.

In another aspect, there is provided the use of polymer as defined above or the hydrogel as defined above or the composition as defined above in the manufacture of a medicament for the treatment of obesity, pre-diabetes, diabetes, non-alcoholic fatty liver disease (NAFLD), or chronic idiopathic constipation, or for reducing caloric intake or improving glycemic control.

Advantageously, the superior mechanical strength and media uptake ratio of the polymer or hydrogel as defined above may be useful in administering to a subject, so that the hydrogel swells in the stomach and reduce caloric intake, improve glycemic control or improve bowel movement, in order to treat obesity, diabetes, non-alcoholic fatty liver disease (NAFLD), or chronic idiopathic constipation.

In another aspect, there is provided a method of weight-loss or improving the body appearance in a healthy subject, comprising the step of orally administering to the subject the polymer as defined above, the hydrogel as defined above or the composition as defined above.

Advantageously, the polymer or hydrogel or composition as defined above may also facilitate non-medical, cosmetic weight-loss to improve the body appearance of a healthy subject.

### Definitions

The following words and terms used herein shall have the meaning indicated:

“Alkyl” as a group or as part of a group refers to a straight or branched aliphatic hydrocarbon group, preferably a C₁-C₆ alkyl, unless otherwise noted. Examples of suitable straight and branched C₁-C₆ alkyl substituents include methyl, ethyl, n-propyl, 2-propyl, n-butyl, sec-butyl, t-butyl, hexyl, and the like. The group may be a terminal group or a bridging group.

“Alkyloxy” refers to an alkyl group as defined herein that is singularly bonded to oxygen. The group may be a terminal group or a bridging group. If the group is a terminal group, it is bonded to the remainder of the molecule through the alkyl group.

“Heteroalkyl” refers to a straight- or branched-chain alkyl group preferably having from 2 to 6 carbons in the chain, one or more of which has been replaced by a heteroatom selected from S, O, P and N. Exemplary heteroalkyls include alkyl ethers, secondary and tertiary alkyl amines, amides, alkyl sulfides, and the like. Examples of heteroalkyl also include hydroxyC₁-C₆alkyl, C₁-C₆alkyloxyC₁-C₆alkyl, aminoC₁-C₆alkyl, C₁-C₆alkylaminoC₁-C₆alkyl, and di(C₁-C₆alkyl)aminoC₁-C₆alkyl. The group may be a terminal group or a bridging group.

“Heterocycloalkyl” refers to a saturated monocyclic, bicyclic, or polycyclic ring containing at least one heteroatom selected from nitrogen, sulfur, oxygen, preferably from 1 to 3 heteroatoms in at least one ring. Each ring is preferably from 3 to 10 membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane, and 1,4-oxathiapane. A heterocycloalkyl group typically is a C₁-C₁₂ heterocycloalkyl group. A heterocycloalkyl group may comprise 3 to 8 ring atoms. A heterocycloalkyl group may comprise 1 to 3 heteroatoms independently selected from the group consisting of N, O and S. The group may be a terminal group or a bridging group.

The term “optionally substituted” as used herein means the group to which this term refers may be unsubstituted, or may be substituted with one or more groups independently selected from acyl, alkyl, alkenyl, alkynyl, thioalkyl, cycloalkyl, cycloalkylalkyl, cycloalkenyl, cycloalkylalkenyl, heterocycloalkyl, cycloalkylheteroalkyl, cycloalkyloxy, cycloalkenyloxy, cycloamino, halo, carboxyl, haloalkyl, haloalkynyl, alkynyloxy, heteroalkyl, heteroalkenyl heteroalkynyl, heteroalkyloxy, hydroxyl, hydroxyalkyl, alkoxy, thioalkoxy, alkenyloxy, haloalkoxy, haloalkenyl, haloalkynyl, haloalkenyloxy, nitro, amino, nitroalkyl, nitroalkenyl, nitroalkynyl, nitroheterocyclyl, alkylamino, dialkylamino, alkenylamine, aminoalkyl, alkynylamino, acyl, alkyloxy, alkyloxyalkyl, alkyloxyaryl, alkyloxycarbonyl, alkyloxycycloalkyl, alkyloxyheteroaryl, alkyloxyheterocycloalkyl, alkenoyl, alkynoyl, acylamino, diacylamino, acyloxy, alkylsulfonyloxy, heterocyclic, heterocycloalkenyl, heterocycloalkyl, heterocycloalkylalkyl, heterocycloalkylalkenyl, heterocycloalkylalkenyl, heterocycloalkylheteroalkyl, heterocycloalkyloxy, heterocycloalkenyloxy, heterocycloxy, heterocycloamino, haloheterocycloalkyl, alkylsulfinyl, alkylsulfonyl, alkylsulfenyl, alkylcarbonyloxy, alkylthio, acylthio, aminosulfonyl, phosphorus-containing groups such as phosphono and phosphinyl, sulfinyl, sulfinylamino, sulfonyl, sulfonylamino, aryl, arylalkyl, arylalkyloxy, arylamino, Arylheteroalkyl, heteroaryl, heteroarylalkyl, heteroarylalkenyl, heteroarylalkynyl, heteroarylheteroalkyl, heteroarylamino, heteroaryloxy, arylalkenyl, arylalkyl, alkylaryl, alkylheteroaryl, aryloxy, arylsulfonyl, cyano, cyanate, isocyanate, —C(O)NH(alkyl), and —C(O)N(alkyl)₂.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### DETAILED DISCLOSURE OF OPTIONAL EMBODIMENTS

There is provided a polymer comprising a polysaccharide crosslinked with a spacer crosslinker, wherein the spacer crosslinker comprises a first optionally substituted aliphatic moiety terminated at each end with a second moiety comprising at least two carboxylic acid groups.

Hydrogels are obtained by physical or chemical stabilization of aqueous solutions of polymeric fiber. Physical stabilization can be achieved via hydrogen bonds, hydrophobic interactions, and chain entanglements. These interactions are generally reversible, and hence hydrogels resulting from polymers that comprise mainly physical interactions may easily flow or degrade. In contrast, chemical crosslinks consist of covalent chemical bonds, and hydrogels formed using polymers comprising chemical crosslinks, such as that defined above, form generally more stable and stiff networks. The degree of crosslinking and type of crosslinker used affects the physical properties of the resulting hydrogel, such as the degree of water retention, mechanical strength and degradation rate.

The spacer crosslinker may have the following formula (I):

A-L-Z-L-A (1)
wherein
Z is the first optionally substituted aliphatic moiety;
A is the second moiety comprising at least two carboxylic acid groups; and
L is a linking group.

The first optionally substituted aliphatic moiety or Z may be derived from a first optionally substituted aliphatic molecule comprising at least two hydroxy groups. In this context, “derived” means that the first optionally substituted aliphatic moiety is formed as a result of the at least two hydroxyl groups of the first optionally substituted aliphatic molecule reacting with the second molecule as defined further below to form part of the linker L in formula (I).

The first optionally substituted aliphatic molecule may be a linear molecule and be terminated at each end with a hydroxyl group.

The first optionally substituted aliphatic molecule may have a molecular weight in the range of about 0.1 kDa to about 100 kDa, about 0.1 kDa to about 0.2 kDa, about 0.1 kDa to about 0.5 kDa, about 0.1 kDa to about 1 kDa, about 0.1 kDa to about 2 kDa, about 0.1 kDa to about 5 kDa, about 0.1 kDa to about 10 kDa, about 0.1 kDa to about 20 kDa, about 0.1 kDa to about 50 kDa, about 0.2 kDa to about 0.5 kDa, about 0.2 kDa to about 1 kDa, about 0.2 kDa to about 2 kDa, about 0.2 kDa to about 5 kDa, about 0.2 kDa to about 10 kDa, about 0.2 kDa to about 20 kDa, about 0.2 kDa to about 50 kDa, about 0.2 kDa to about 100 kDa, about 0.5 kDa to about 1 kDa, about 0.5 kDa to about 2 kDa, about 0.5 kDa to about 5 kDa, about 0.5 kDa to about 10 kDa, about 0.5 kDa to about 20 kDa, about 0.5 kDa to about 50 kDa, about 0.5 kDa to about 100 kDa, about 1 kDa to about 2 kDa, about 1 kDa to about 5 kDa, about 1 kDa to about 10 kDa, about 1 kDa to about 20 kDa, about 1 kDa to about 50 kDa, about 1 kDa to about 100 kDa, about 2 kDa to about 5 kDa, about 2 kDa to about 10 kDa, about 2 kDa to about 20 kDa, about 2 kDa to about 50 kDa, about 2 kDa to about 100 kDa, about 5 kDa to about 10 kDa, about 5 kDa to about 20 kDa, about 5 kDa to about 50 kDa, about 5 kDa to about 100 kDa, about 10 kDa to about 20 kDa, about 10 kDa to about 50 kDa, about 10 kDa to about 100 kDa, about 20 kDa to about 50 kDa, about 20 kDa to about 100 kDa, or about 50 kDa to about 100 kDa.

Using a long hydrophilic spacer crosslinker (such as one having the molecular weight as defined above) allows for the formation of a polymer with a looser polymeric network while still achieving a high level of strength, as measured by the swelled state tensile modulus. A looser polymeric network leads to a greater swelling ratio of the hydrogel as it allows the polysaccharide chains within the network to move further away from each other, allowing for the polymer network to swell to a greater extent.

When a short crosslinker such as citric acid is used, two polysaccharide chains can be connected at a distance via third polysaccharide chain linking the two chains. However, the length of the connection is random. Therefore, generally, the linker length determines the proximity of the connected polysaccharide chains. Since multiple crosslinkers can be attached to a single chain at random points, using a short crosslinker results in a polymeric network in which the polysaccharide chains are linked close together, leading to a compacted network. In contrast, when long hydrophilic crosslinkers are used, the distance between two polysaccharide chains will be determined by the length of the long hydrophilic crosslinker. Since the polysaccharide chains will be connected to each other via a fixed chain length corresponding to the length of the long hydrophilic crosslinker, using a long hydrophilic crosslinker will lead to a looser polymeric network.

The strength of the hydrogel depends on the degree of interaction between the polymeric chains. When a short crosslinker such as citric acid is used, once one end of the crosslinker reacts with a polysaccharide chain, the other end can only react within the same polysaccharide chain, or with another polysaccharide chain that is in close proximity with the first polysaccharide chain, due to its short length. This severely limits the crosslinking network that can be formed. The mobility of a short crosslinker that has reacted on one end with a polysaccharide chain is low, as the polysaccharide chain is itself long and relatively immobile. This limited mobility prevents the other end of the crosslinker to move around, and therefore results in crosslinks to form within the same polysaccharide chain or with a second polysaccharide chain that is already crosslinked to the first polysaccharide chain, since they are already in close proximity to each other. This is undesirable, as intramolecular crosslinking decreases the swelling ratio without a significant increase in tensile modulus.

In contrast, if long hydrophilic crosslinkers are used, when one end of the crosslinker reacts with a polysaccharide chain, the other end can move around and react with a polysaccharide chain which is significantly further away from the first polysaccharide chain, due to the flexible nature of the long crosslinker. Therefore, when a long hydrophilic crosslinker is used, it is highly likely that the second polysaccharide chain will be a different chain which has not been crosslinked to the first polysaccharide chain. This overcomes the limitations of low mobility observed when using a short crosslinker. Further, since it is unlikely for crosslinking to occur within the same polysaccharide chain or to occur multiple times between two polysaccharide chains, the amount of crosslinkers used can be reduced, while still retaining a strong polymeric network structure.

The first optionally substituted aliphatic molecule may be saturated or unsaturated, linear or branched.

The first optionally substituted aliphatic molecule may comprise an optionally substituted alkyl or optionally substituted heteroalkyl. The optionally substituted alkyl may be optionally substituted with a substituent selected from the group consisting of hydroxyl, alkyloxy, carboxyl, thioalkoxy and carboxyamide. The optionally substituted heteroalkyl may be an ether or amine.

The first optionally substituted aliphatic molecule may be a hydrophilic polymer.

The first optionally substituted aliphatic molecule may be selected from the group consisting of polyether, polyacrylamide, polyethyleneimine, polyacrylate, polymethacrylate, polyvinyl pyrrolidone and polyvinyl alcohol, each further comprising at least two hydroxy groups.

The first optionally substituted aliphatic moiety or Z may have the following structure

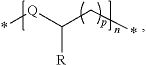

wherein:
Q is —CH₂—, —O— or —NH₂—,
R is hydrogen, —OH, optionally substituted C₁ to C₆ alkyl, —C(O)OM, —C(O)NR²R³, or optionally substituted heterocycloalkyl,
R² and R³ are independently hydrogen or optionally substituted C₁ to C₆ alkyl,
M is R², Na or K,
p in an integer in the range of 1 to 6,
n is an integer in the range of 2 to 2000, and
* indicates where the moiety attaches to the rest of the spacer crosslinker.

R may be hydrogen, methyl, ethyl, propyl, butyl, pentyl or hexyl. R may be hydrogen or methyl.

R may be —C(O)OH, —C(O)ONa or —C(O)OK.

The heteroatom of the optionally substituted heterocycloalkyl may be N.

The optionally substituted heterocycloalkyl may comprise a heteroatom N and may be bonded to the rest of the optionally substituted aliphatic moiety via the N atom.

R may be selected from the group consisting of 2-pyrrolidone, 3-pyrrolidone, pyrrolidine, imidazolidine, pyrazolidine, piperidine, morpholine and diazine.

R may be C(O)NR²R³, and when R is C(O)NR²R³, R² and R³ may both be hydrogen.

p may be an integer of 1, 2, 3, 4, 5 or 6.

n may be an integer in the range of 2 to 5, 2 to 10, 2 to 20, 2 to 50, 20 to 100, 2 to 200, 2 to 500, 2 to 1000, 2 to 2000, 5 to 10, 5 to 20, 5 to 50, 5 to 100, 5 to 200, 5 to 500, 5 to 1000, 5 to 2000, 10 to 20, 10 to 50, 10 to 100, 10 to 200, 10 to 500, 10 to 1000, 10 to 2000, 20 to 50, 20 to 100, 20 to 200, 20 to 500, 20 to 1000, 20 to 2000, 50 to 100, 50 to 200, 50 to 500, 50 to 1000, 50 to 2000, 100 to 200, 100 to 500, 100 to 1000, 100 to 2000, 200 to 500, 200 to 1000, 200 to 2000, 500 to 1000, 500 to 2000 or 1000 to 2000.

The first optionally substituted aliphatic moiety or Z may have the following structure

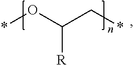

wherein
R is hydrogen or an optionally substituted C₁ to C₆ alkyl,
n is an integer in the range of 2 to 2000, and
* indicates where the moiety attaches to the rest of the spacer crosslinker.

The first optionally substituted aliphatic molecule may be polyethylene glycol or polypropylene glycol each further comprising at least two hydroxy groups.

Polyethylene glycol (PEG) is a polyether that is amphiphilic and soluble in water as well in many organic solvents. PEG is readily available in a wide range of molecular weights and it has been found to be nontoxic and is approved by the US Food and Drug Administration (FDA). Modified PEG having a low polydispersity index and reactive groups at both ends can be used as a long hydrophilic crosslinker to prepare hydrogels with different physical properties depending on the PEG chain length used.

The second moiety or A comprising at least two carboxylic acid groups may be derived from a second molecule having at least three carboxylic acid groups. In this context, “derived” means that the second moiety comprising at least two carboxylic acid groups is formed when one of the carboxylic acid groups of the second molecule having at least three carboxylic acid groups is reacted to form part of the linker L in formula (I).

Two of the at least two carboxylic acid groups in the second moiety or A may be separated by 2 to 6 atoms, 2 to 3 atoms, 2 to 4 atoms, 2 to 5 atoms, 3 to 4 atoms, 3 to 5 atoms, 3 to 6 atoms, 4 to 5 atoms, 4 to 6 atoms or 5 to 6 atoms. Advantageously, by having two of the at least two carboxylic acid groups separated by 2 to 6 atoms, the second moiety or A may form a cyclic anhydride intermediate, which may act as an intramolecular catalyst during the crosslinking process between the spacer crosslinker and the polysaccharide.

The second molecule having at least three carboxylic acid groups may be selected from the group consisting of citric acid, pyromellitic acid, butanetetracarboxylic acid, and benzoquinonetetracarboxylic acid.

The second moiety or A comprising at least two carboxylic acid groups may be selected from the group consisting of:

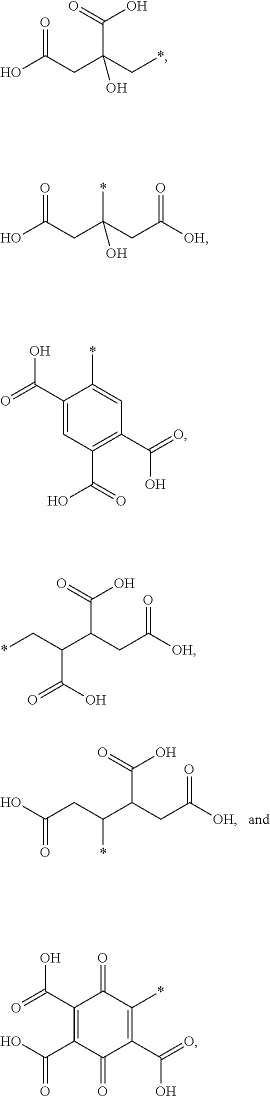

wherein * indicates where the moiety attaches to the rest of the spacer crosslinker.

L may be independently selected from the group consisting of an amide, ester, acid anhydride and thioester.

The polysaccharide may be selected from the group consisting of starch, cellulose, galactomannan and alginate.

Given the growing concern on environmental protection, recent interest has focused on the development of superabsorbent hydrogels based on biodegradable materials having properties which are similar to traditional but non-biodegradable superabsorbent polyacrylates. Suitable biodegradable polymers include polysaccharides, such as alginate, starch, and cellulose derivatives.

The polysaccharide may comprise at least one carboxymethyl group.

The polysaccharide may be carboxylmethylcellulose.

Carboxymethylcellulose (CMC) or cellulose gum is a cellulose derivative with carboxymethyl groups (—CH₂—COOH) bound to some of the hydroxyl groups of the glucopyranose monomers that make up the cellulose backbone. CMC can be synthesized by the alkali-catalysed reaction of cellulose with chloroacetic acid. This reaction is followed by a purification process to produce pure CMC for use in food, pharmaceutical, and dentifrice (toothpaste) applications.

CMC can be used in food as a viscosity modifier or thickener, and to stabilize emulsions in various products including ice cream. It is also a constituent of many non-food products, such as toothpaste, laxatives, diet pills, water-based paints, detergents, textile sizing, reusable heat packs, and various paper products. It is used primarily because it has high viscosity, is nontoxic, and is generally considered to be hypoallergenic, since the major source of fiber is either softwood pulp or cotton linter.

The carboxymethylcellulose may have a degree of substitution in the range of about 0.6 to about 1.0, about 0.6 to about 0.8 or about 0.8 to about 1.0.

The functional properties of CMC may depend on the degree of substitution of the cellulose structure, as well as the chain length of the cellulose backbone structure and the degree of clustering of the carboxymethyl substituents. A degree of substitution in the range of about 0.6 to about 1.0 allows for better emulsifying properties and improves resistance to acids and salts.

The polysaccharide may have a viscosity, as a 1% (wt/wt) aqueous solution at 25° C., of greater than about 1000 cps, greater than about 2000 cps, greater than about 3000 cps, greater than about 5000 cps, greater than about 7000 cps, or greater than about 10,000 cps. The polysaccharide may have a viscosity, as a 1% (wt/wt) aqueous solution at 25° C., in the range of about 1000 cps to about 12000 cps, about 1000 cps to about 5000 cps, about 1000 cps to about 10,000 cps, about 5000 cps to about 10,000 cps, about 5,000 cps to about 12,000 cps or about 10,000 cps to about 12,000 cps.

The polysaccharide molecular weight may have a polydispersity index of less than 10, less than 5 or less than 2. The polysaccharide may have a polydispersity index, in the range of about 1 to about 10.

The polymer may be in the form of a powder having a particle size in the range of about 0.05 mm to about 5 mm, about 0.05 mm to about 0.1 mm, about 0.05 mm to about 2 mm, about 0.1 mm to about 0.2 mm, about 0.1 mm to about 0.5 mm, about 0.1 mm to about 1 mm, about 0.1 mm to about 2 mm, about 0.1 mm to about 5 mm, about 0.2 mm to about 0.5 mm, about 0.2 mm to about 0.5 mm, about 0.2 mm to about 1 mm, about 0.2 mm to about 2 mm, about 0.2 mm to about 5 mm, about 0.5 mm to about 1 mm, about 0.5 mm to about 2 mm, about 0.5 mm to about 5 mm, about 1 mm to about 2 mm, about 1 mm to about 5 mm or about 2 mm to about 5 mm.

The polymer as defined above may be biodegradable. The polymer as defined above may comprise carboxymethylcellulose as the polysaccharide, a first optionally substituted aliphatic moiety derived from polyethylene glycol terminated at each end with a hydroxyl group and a second moiety derived from citric acid. Each of carboxymethylcellulose, polyethylene glycol and citric acid may be independently biodegradable and as such, the resulting polymer may also be biodegradable.

There is also provided a hydrogel comprising the polymer as defined above and a liquid.

The liquid may be an aqueous liquid. The liquid may be water, buffer, gastric fluid, simulated gastric fluid or any mixture thereof.

The hydrogel may have a rheological property as measured by the G′ value in the range of about 500 Pa to about 10,000 Pa, about 500 Pa to about 1000 Pa, 500 Pa to about 2000 Pa, about 500 Pa to about 5000 Pa, about 1000 Pa to about 2000 Pa, about 1000 Pa to about 5000 Pa, about 1000 Pa to about 10,000 Pa, about 2000 Pa to about 5000 Pa, about 2000 Pa to about 10,000 Pa or about 5000 Pa to about 10,000 Pa.

The hydrogel may have a media uptake ratio (MUR) of at least 50, at least 70, at least 90, or at least 100. The hydrogel may have a media uptake ratio in the range of about 50 to about 200.

At least about 70% by mass, about 80% by mass or about 90% by mass or 100% by mass of the hydrogel may comprise the polymer in the form of particles in the size range of about 0.1 mm to about 2 mm.

The hydrogel may have a tape density in the range of about 0.2 g/mL to about 2.0 g/mL, about 0.2 g/mL to about 0.5 g/mL, about 0.2 g/mL to about 1.0 g/mL, about 0.5 g/mL to about 1.0 g/mL, about 0.5 g/mL to about 2.0 g/mL or about 1.0 g/mL to about 2.0 g/mL.

The hydrogel may have a loss on drying of about 20% (wt/wt) or less, about 10% (wt/wt) or less, about 5% (wt/wt) or less, about 2% (wt/wt) or less or about 1% (wt/wt) or less. The hydrogel may have a loss of drying in the range of about 0.1% (wt/wt) to about 20% (wt/wt).

The hydrogel may have a G′ value in the range of about 500 Pa to 10000 Pa and a media uptake ratio of at least 50, when determined on a sample of the polymer in the form of particles whereby at least 80% by mass of the particles are in the size range of 0.1 mm to 2 mm, having a tape density in the range of 0.5 g/mL to 1.0 g/mL and a loss on drying of 10% (wt/wt) or less.

There is also provided a method of forming the polymer as defined above, comprising the steps of:
a) reacting a first optionally substituted aliphatic molecule comprising at least two hydroxyl groups with a second molecule comprising at least three carboxylic acid groups to form a spacer crosslinker; and
b) crosslinking the spacer crosslinker with a polysaccharide to form the polymer as defined above.

The reacting step (a) may further comprise a polymer additive.

The polymer additive may be a hydrophilic molecule which may be added to the mixture of a first optionally substituted aliphatic molecule comprising at least two hydroxyl groups with a second molecule comprising at least three carboxylic acid groups to form a spacer crosslinker before the crosslinking step, to confer additional properties to the polymer or hydrogel, such as to increase the rate at which the resulting polymer or hydrogel swells. The polymer additive may at least be partially crosslinked with the polymer. The polymer additive may be substantially not crosslinked with the polymer. The polymer additive may not be crosslinked with the polymer.

The polymer may comprise the polysaccharide as defined above crosslinked with the spacer crosslinker as defined above and at least partially crosslinked with the polymer additive as defined above.

The polymer may comprise the polysaccharide as defined above crosslinked with the spacer crosslinker as defined above, but the additive as defined above may not be crosslinked with the polymer.

The polymer additive may be a plasticizer. The polymer additive may be a hydrophilic oligomer such as polyethylene glycol (PEG). Advantageously, PEG may be highly hydrophilic, may hinder the entanglement of the spacer crosslinker, and may be easily added into the polymer without affecting the number of reactive hydroxyl groups per unit mass.

When the crosslinker as defined above is used in the crosslinking reaction of the polymer, the crosslinkers may become entangled with each other, which may restrict the mobility of the polymer's dimensional network. Polymer additives such as a plasticizer may prevent the entanglement of the crosslinker during the crosslinking process, thereby ensuring higher mobility of the polymer's dimensional network, and therefore better swelling rates.

The hydrophilic oligomer may be selected from the group consisting of PEG-100, PEG-200, PEG-400, PEG-1000 and any mixture thereof.

The reacting step (a) may further comprise a catalyst. The catalyst may be selected from the group consisting of ammonia, ammonium sulfate, aluminium sulfate, magnesium chloride, magnesium acetate, zinc chloride, zinc nitrate and any mixture thereof or the catalyst may comprise phosphorus.

The catalyst may be sodium phosphate, sodium hypophosphite or a (1:1) mixture by weight of sodium bicarbonate combined with disodium phosphate.

The reacting step (a) may be performed in the absence of a catalyst.

The reacting step (a) and crosslinking step (b) may be independently performed at a temperature in the range of about 80° C. to about 180° C., about 80° C. to about 100° C., about 80° C. to about 130° C., about 80° C. to about 150° C., about 100° C. to about 130° C., about 100° C. to about 150° C., about 100° C. to about 180° C., about 130° C. to about 150° C., about 130° C. to about 180° C., or about 150° C. to about 180° C.

During the crosslinking step (b), the ratio by weight of the second molecule comprising at least three carboxylic acid groups to the polysaccharide may be less than about 1:30, less than about 1:50, less than about 1:100, less than about 1:500. The ratio by weight of the second molecule comprising at least three carboxylic acid groups to the polysaccharide may be in the range of about 1:30 to about 1:50, 1:30 to about 1:100, about 1:30 to about 1:500, about 1:30 to about 1:1000, about 1:50 to about 1:100, about 1:50 to about 1:500, about 1:50 to about 1:1000, about 1:100 to about 1:500, about 1:100 to about 1:500, or about 1:500 to about 1:1000.

The method may further comprise the following steps (a1), (a2) and (a3) between the reacting step (a) and the crosslinking step (b):
a1) mixing the spacer crosslinker and the polysaccharide in a solvent to form a homogenized mixture,
a2) drying the homogenized mixture at a temperature in the range of about 40° C. to about 90° C. to remove the solvent, and
a3) grinding the dried homogenized mixture to form a powder having a particle size in the range of about 0.05 mm to about 5 mm.

The mixing step a1) may further comprise the polymer additive as defined above.

The drying step of the homogenized mixture may be performed at a temperature in the range of about 40° C. to about 90° C., about 40° C. to about 60° C. or about 60° C. to about 90° C.

The powder of the dried homogenized mixture may have a particle size in the range of about 0.05 mm to about 5 mm, about 0.05 mm to about 0.1 mm, about 0.05 mm to about 2 mm, about 0.1 mm to about 0.2 mm, about 0.1 mm to about 0.5 mm, about 0.1 mm to about 1 mm, about 0.1 mm to about 2 mm, about 0.1 mm to about 5 mm, about 0.2 mm to about 0.5 mm, about 0.2 mm to about 0.5 mm, about 0.2 mm to about 1 mm, about 0.2 mm to about 2 mm, about 0.2 mm to about 5 mm, about 0.5 mm to about 1 mm, about 0.5 mm to about 2 mm, about 0.5 mm to about 5 mm, about 1 mm to about 2 mm, about 1 mm to about 5 mm or about 2 mm to about 5 mm.

The method may further comprise the step of washing the polymer with deionized water after the crosslinking step (b). The washing step of the polymer may be performed for a duration in the range of a about 2 hours to about 36 hours, about 2 hours to about 6 hours, about 2 hours to about 12 hours, about 2 hours to about 18 hours, about 2 hours to about 24 hours, about 6 hours to about 12 hours, about 6 hours to about 18 hours, about 6 hours to about 24 hours, about 12 hours to about 18 hours, about 12 hours to about 24 hours, about 12 hours to about 36 hours, about 18 hours to about 24 hours, about 18 hours to about 36 hours or about 24 hours to about 36 hours During the washing step, the washing solution (deionized water) may be changed 1, 2, 3, 4 or 5 times to remove any impurities.

The method may further comprise the step of drying the polymer after the washing step. The drying of the polymer may be performed at a temperature in the range of about 40° C. to about 90° C., about 40° C. to about 60° C. or about 60° C. to about 90° C. The drying of the polymer may be performed for a duration in the range of about 6 hours to about 36 hours, about 6 hours to about 12 hours, about 6 hours to about 18 hours, about 6 hours to about 24 hours, about 12 hours to about 18 hours, about 12 hours to about 24 hours, about 12 hours to about 36 hours, about 18 hours to about 24 hours, about 18 hours to about 36 hours or about 24 hours to about 36 hours.

The method may further comprise the step of grinding the polymer after the drying step of the polymer to form a powder having a particle size in the range of about 0.05 mm to about 5 mm.

The method may further comprise a step of adding a liquid to the polymer.

The crosslinked polymer may be considered a hydrogel in the presence of a liquid.

There is also provided a polymer obtained by the method as defined above.

There is also provided a hydrogel obtained by the method as defined above, when the polymer is in contact with a liquid.

There is also provided a composition comprising the polymer as defined above or the hydrogel as defined above and a pharmaceutically acceptable excipient.

The polymer composition may further comprise a polymer additive as defined above.

The polymer or hydrogel can be administered alone. Alternatively, the polymer or hydrogel can be administered as a pharmaceutical, veterinarial, or industrial formulation. The polymer or hydrogel can also be present as suitable salts, including pharmaceutically acceptable salts.

The language “pharmaceutically acceptable excipient” is intended to include, but is not limited to, solvents, dispersion media, coatings, anti-bacterial and anti-fungal agents, isotonic and absorption delaying agents, and the like. The use of such media and agents for pharmaceutically active substances is well known in the art. Except insofar as any conventional media or agent is incompatible with the polymer or hydrogel, use thereof in the therapeutic compositions and methods of treatment and prophylaxis is contemplated. Supplementary active compounds may also be incorporated.

It is especially advantageous to formulate parenteral compositions in dosage unit form for ease of administration and uniformity of dosage. “Dosage unit form” as used herein refers to physically discrete units suited as unitary dosages for the individual to be treated; each unit containing a predetermined quantity of polymer or hydrogel is calculated to produce the desired therapeutic effect in association with the required pharmaceutical excipient. The polymer or hydrogel may be formulated for convenient and effective administration in effective amounts with a suitable pharmaceutically acceptable excipient in an acceptable dosage unit. In the case of compositions containing supplementary active ingredients, the dosages are determined by reference to the usual dose and manner of administration of the said ingredients.

The excipient can be selected from, but is not limited to, agents such as gum gragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir can contain the analogue, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the analogue may be incorporated into sustained-release preparations and formulations.

In one example, the excipient is an orally administrable excipient.

There is also provided a capsule comprising the polymer as defined above or the hydrogel as defined above.

Each capsule may comprise the polymer as defined above at an amount in the range of about 0.5 g to about 1 g, about 0.5 g to about 0.75 g or about 0.75 g to about 1 g. Each capsule may comprise the polymer as defined above at an amount in the range of about 0.7 g to about 0.8 g.

The capsule may be made of gelatin and may be used for oral administration of the polymer or hydrogel to a subject.

In one example, the polymer or hydrogel is to be administered orally. The polymer or hydrogel can be administered orally, for example, with an inert diluent or an assimilable edible carrier. The polymer or hydrogel and other ingredients can also be enclosed in a hard or soft shell gelatin capsule, compressed into tablets, or incorporated directly into an individual's diet. For oral therapeutic administration, the polymer or hydrogel can be incorporated with excipients and used in the form of ingestible tablets, buccal tablets, troches, capsules, elixirs, suspensions, syrups, wafers, and the like.

There is also provided a method of treating obesity, pre-diabetes, diabetes, non-alcoholic fatty liver disease (NAFLD), or chronic idiopathic constipation, or of reducing caloric intake or improving glycemic control in a subject in need thereof, comprising the step of orally administering to the subject a therapeutically effective amount of the polymer as defined above or the hydrogel as defined above or the composition as defined above.

As used herein the term “treatment”, refers to any and all uses which remedy a disease state or symptoms, prevent the establishment of disease, or otherwise prevent, hinder, retard, or reverse the progression of disease or other undesirable symptoms in any way whatsoever.

One skilled in the art would be able to determine effective, non-toxic dosage levels of the polymer or hydrogel and an administration pattern which would be suitable for treating the diseases or conditions to which the polymer or hydrogel is applicable.

Further, it will be apparent to one of ordinary skill in the art that the optimal course of treatment, such as the number of doses of polymer or hydrogel given per day for a defined number of days, can be ascertained using convention course of treatment determination tests.

The polymer or hydrogel may be administered alone. Alternatively, the polymer or hydrogel may be administered as a pharmaceutical, veterinarial, or industrial formulation. The polymer or hydrogel may also be present as suitable salts, including pharmaceutically acceptable salts.

In one example, the polymer or hydrogel is to be administered orally. The polymer or hydrogel can be administered orally, for example, with an inert diluent or an assimilable edible carrier. The polymer or hydrogel and other ingredients can also be enclosed in a hard or soft shell gelatin capsule, compressed into tablets, or incorporated directly into an individual's diet. For oral therapeutic administration, the polymer or hydrogel can be incorporated with excipients and used in the form of ingestible tablets, buccal tablets, troches, capsules, elixirs, suspensions, syrups, wafers, and the like.

It is especially advantageous to formulate parenteral compositions in dosage unit form for ease of administration and uniformity of dosage. “Dosage unit form” as used herein refers to physically discrete units suited as unitary dosages for the individual to be treated; each unit containing a predetermined quantity of the polymer or hydrogel is calculated to produce the desired therapeutic effect in association with the required pharmaceutical excipient. The polymer or hydrogel may be formulated for convenient and effective administration in effective amounts with a suitable pharmaceutically acceptable excipient in an acceptable dosage unit. In the case of polymer or hydrogel containing supplementary active ingredients, the dosages are determined by reference to the usual dose and manner of administration of the said ingredients.

The dosage unit form may be a solid form, for example, as pellets, tablets, capsules, lozenges, wafers or crackers or in liquid form, for example, as solutions, or emulsions.

The dosage unit form, in solid form, may be further coated with a pharmaceutically acceptable excipient. The coating of a dosage unit form may be carried out in a fluid bed processor using a bottom spray, a top spray, or a tangential spray attachment. The flowability, processability and other characteristics of the dosage unit form may be readily controlled through the choice of appropriate pharmaceutically acceptable excipients onto which the dosage unit forms are coated; and by varying the process variables such as the spray rate and the degree of fluidization.

In one example, the polymer or hydrogel is to be administered in single or multiple doses. In one example, the polymer or hydrogel is to be administered in a single, double, triple or quadruple doses. In another example, the polymer or hydrogel can be, or is to be, administered at an interval of, but not limited to, hourly, daily, twice daily, thrice daily, 4 times a day, every second day, every third day, every fourth day, every fifth day, every sixth day, weekly, biweekly, bimonthly, monthly, or combinations thereof.

Generally, an effective dosage per 24 hours may be in the range of about 0.001 mg to about 500 mg per kg body weight; about 0.001 mg to about 0.01 mg per kg body weight, about 0.001 mg to about 0.1 mg per kg body weight, about 0.001 mg to about 1 mg per kg body weight, about 0.001 mg to about 10 mg per kg body weight, about 0.001 mg to about 100 mg per kg body weight, about 0.01 mg to about 500 mg per kg body weight; about 0.01 mg to about 0.1 mg per kg body weight, about 0.01 mg to about 1 mg per kg body weight, about 0.01 mg to about 10 mg per kg body weight, about 0.01 mg to about 100 mg per kg body weight, about 0.1 mg to about 500 mg per kg body weight; about 0.1 mg to about 1 mg per kg body weight, about 0.1 mg to about 10 mg per kg body weight, about 0.1 mg to about 100 mg per kg body weight, about 1 mg to about 500 mg per kg body weight; about 1 mg to about 10 mg per kg body weight, about 1 mg to about 100 mg per kg body weight, about 10 mg to about 500 mg per kg body weight; about 10 mg to about 100 mg per kg body. More suitably, an effective dosage per 24 hours may be in the range of about 10 mg to about 500 mg per kg body weight; about 10 mg to about 250 mg per kg body weight; about 50 mg to about 500 mg per kg body weight; about 50 mg to about 200 mg per kg body weight; or about 50 mg to about 100 mg per kg body weight.

An effective dosage routine may be once a week, twice a week, thrice a week, daily, twice daily or thrice daily.

An effective dosage routine may be twice or thrice daily, and each dose may comprise one, two, three, four or five dosage unit forms as defined above.

Each dose may comprise about 1 g to about 6 g, about 1 g to about 2 g, about 1 g to about 3 g, about 1 g to about 4 g, about 1 g to about 5 g, about 2 g to about 3 g, about 2 g to about 3 g, about 2 g to about 4 g, about 2 g to about 5 g, about 2 g to about 5 g, about 2 g to about 6 g, about 3 g to about 4 g, about 3 g to about 5 g, about 3 g to about 6 g, about 4 g to about 5 g, about 4 g to about 6 g, or about 5 g to about 6 g of the polymer or hydrogel as defined above.

Each dose may comprise 2 to 8 dosage unit forms, 2 to 3 dosage unit forms, 2 to 4 dosage unit forms, 2 to 5 dosage unit forms, 2 to 6 dosage unit forms, 2 to 7 dosage unit forms, 3 to 4 dosage unit forms, 3 to 5 dosage unit forms, 3 to 6 dosage unit forms, 3 to 7 dosage unit forms, 3 to 8 dosage unit forms, 4 to 5 dosage unit forms, 4 to 6 dosage unit forms, 4 to 7 dosage unit forms, 4 to 8 dosage unit forms, 5 to 6 dosage unit forms, 5 to 7 dosage unit forms, 5 to 8 dosage unit forms, 6 to 7 dosage unit forms, 6 to 8 dosage unit forms or 7 to 8 dosage unit forms comprising the polymer or hydrogel as defined above.

Each dose may comprise about 2.24 g of the polymer or hydrogel as defined above, administered as 4 dosage unit forms, wherein each dosage unit form in the form of a capsule may comprise about 0.56 g of the polymer or hydrogel as defined above.

The polymer or hydrogel may be administered before a meal. The polymer or hydrogel may be administered about 10 minutes to about 1 hour, about 10 minutes to about 20 minutes, about 10 minutes to about 30 minutes, about 10 minutes to about 45 minutes, about 20 minutes to about 30 minutes, about 20 minutes to about 45 minutes, about 20 minutes to about 1 hour, about 30 minutes to about 45 minutes, about 30 minutes to about 1 hour or about 45 minutes to about 1 hour before a meal.

The polymer or hydrogel may be administered with water. The polymer or hydrogel may be administered with about 100 mL to about 700 mL, about 100 mL to about 250 mL, about 100 mL to about 500 mL, about 250 mL to about 500 mL, about 250 mL to about 700 mL or about 500 mL to about 700 mL of water.

The polymer or hydrogel of the invention can be used in combination with other known treatments for the disease or condition. Combinations of active agents, including the polymer or hydrogel, can be synergistic.

The subject can be, but is not limited to, an animal that is as risk or is suffering from obesity, pre-diabetes, diabetes, non-alcoholic fatty liver disease (NAFLD), or chronic idiopathic constipation. The subject may further be in the need to reduce caloric intake or improve glycemic control. In one example, the animal is a human.

There is also provide the polymer as defined above or the hydrogel as defined above or the composition as defined above for use in the treatment of obesity, pre-diabetes, diabetes, non-alcoholic fatty liver disease (NAFLD), or chronic idiopathic constipation, or for reducing caloric intake or improving glycemic control.

There is also provide the use of polymer as defined above or the hydrogel as defined above or the composition as defined above in the manufacture of a medicament for the treatment of obesity, pre-diabetes, diabetes, non-alcoholic fatty liver disease (NAFLD), or chronic idiopathic constipation, or for reducing caloric intake or improving glycemic control.

There is also provided a method of weight-loss or a method of improving the body appearance in a healthy subject, comprising the step of orally administering to the subject the polymer as defined above or the hydrogel as defined above or the composition as defined above.

The method of weight-loss or a method of improving the body appearance may be purely cosmetic.

The amount of polymer or hydrogel or composition administered in a method of weight-loss or a method of improving the body appearance may be the same as the amount of polymer or hydrogel or composition administered in the method of treating obesity as defined above.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 shows a schematic diagram showing the synthetic flow of preparing the spacer crosslinkers and crosslinked CMC hydrogels. (101) indicates the reaction to prepare the spacer crosslinkers, and (102) indicates the reaction to prepare the crosslinked carboxymethylcellulose hydrogels.

FIG. 2 shows a schematic diagram of the locations of the intradermal injection sites in the skin sensitization test. (202) indicates the cranial end, (204) indicates the caudal end, (206) indicates 0.1 mL intradermal injection sites, and (208) indicates the clipped intrascapular region.

FIG. 3 shows a schematic diagram comparing the crosslinking mechanism of CMC hydrogel examples and controls. (302) indicates the reaction to form control C-1, (304) indicates the reaction to form example 7-13 and (306) indicates the reaction to form control C-2. (310) indicates CMC, (312) indicates CA, (314) indicates PEG-CA and (316) indicates PEG.

### EXAMPLES

Non-limiting examples of the invention will be further described in greater detail by reference to specific Examples, which should not be construed as in any way limiting the scope of the invention.

### Materials

Carboxymethylcellulose (CMC) sodium salt was obtained from AQUALON™ 7H3SF (Ashland Inc.), which has a viscosity of 1,000 to 2,800 cps as a 1% (wt/wt) solution in water at 25° C. Polyethylene glycol (PEG, average molecular weight ˜200, 400, 1K, 2K, 4K, 8K) was purchased from Sigma-Aldrich and used without further modification. Citric acid (CA) was obtained from Tokyo Chemical Industry (TCI) and used without further modification. Chemicals including sodium hypophosphite (SHP), sodium bicarbonate, disodium phosphate, sodium chloride (NaCl), and sodium hydroxide (NaOH), hydrochloric acid (HCl), were purchased from Sigma-Aldrich and used as received. Sodium bicarbonate combined with disodium phosphate (1:1 by weight) formed dual catalysts (CAT2) for esterification. Unless specified otherwise, deionized (DI) water with a resistivity of 18.2 M ohm-cm was used to through all experiments and the procedures were performed at room temperature (23±2° C.).

### Characterization Methods

Esterification Between PEG with CA (Calculation Method A)

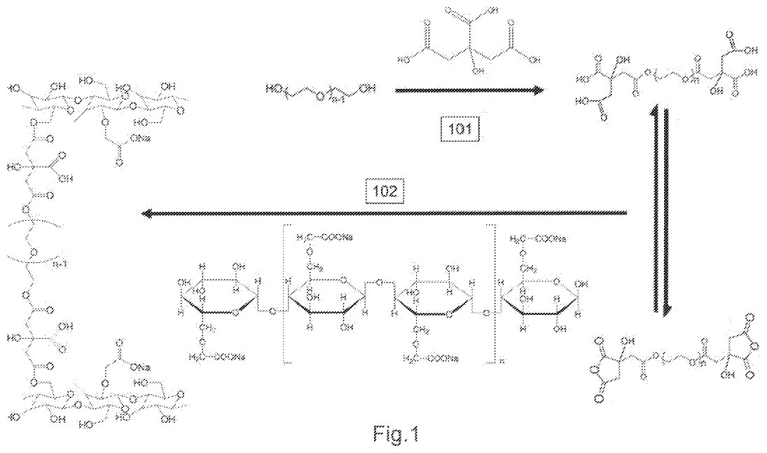

Base titration was used to determine the successful synthesis of spacer crosslinkers. After the Step 1 reaction (FIG. 1, (101)), 3 mL (equivalent to 100 mg CA) of the resulting solution was diluted with DI water to 50 mL. A few drops of phenolphthalein ethanol (1:100) solution was added into the crosslinker solution, then titrated with 0.1N NaOH until the total solution colour changed from clear to pink. Consumed volume of NaOH was recorded and compared with controls which directly mixed same amount of PEG and CA. For example:
CA initial input fixed at 1 g, and equivalent concentration of COOH=1000/192*3=15.6 mmol;
PEG200 input fixed at 0.5 g, equivalent concentration of OH=500/200*2=5 mmol;
PEG400, 1000, 2000 and 4000 initial input fixed at 1 g, 2.5 g, 5 g and 10 g, respectively;
In theory, 100% esterification corresponding to concentration of COOH reduction %=5/15.6=32%;
The real esterification degree can be estimated via real concentration of COOH reduction % divided by 32%.

### Equilibrium Swelling

Media uptake measurements were performed on samples of the dried crosslinked CMC in powder form (100-1000 microns particle size distribution) soaked for 30 minutes in different media. Standard Simulated Gastric Fluid (SGF) was prepared by mixing 7 mL HCl 37%, 2 g NaCl and 3.2 g pepsin in DI water. After solid dissolution, more water was added to achieve a volume of 1 L. Diluted SGF (Di-SGF) was prepared by mixing 1 part SGF with 8 parts DI water, then simulating gastric fluid after water intake with pills/capsules containing the dried crosslinked CMC.

Media uptake ratio (MUR) of crosslinked hydrogels in Di-SGF was determined as follows: A dried glass funnel was placed on a support and 40 g of purified water was poured into the funnel. Once no further droplets were detected in the neck of the funnel (about 5 minutes), the funnel was placed into an empty and dry glass beaker (beaker #1), which was placed on a tared scale to record the weight of the empty apparatus (W1). 40 g of DI-SGF solution was prepared as described above and placed in beaker #2. 0.25 g of crosslinked carboxymethylcellulose powder was accurately weighed using weighing paper. The carboxymethylcellulose powder was added to beaker #2 and stirred gently for 30 minutes with a magnetic stirrer without generating vortices. The stir bar was removed from the resulting suspension, the funnel was placed on a support and the suspension was poured into the funnel to allow the material to drain for 10±1 minute. The funnel containing the drained material was placed inside beaker #1 and weighed (W2). The Media Uptake Ratio (MUR) was calculated according to: MUR=(W2−W1)/0.25. The determination was made in triplicate.

### Mechanical Strength

Viscoelastic properties of the polymer hydrogels were determined according to the protocol set below. Hydrogels were freshly prepared according to MUR testing methods described above for equilibrium swelling. Briefly, 0.25 g of crosslinked carboxymethylcellulose powder was soaked with 40 g of DI-SGF solution and stirred for 30 minutes. The swelled suspension was poured into a filtration funnel and drained for 10 minutes, and the resulting hydrogel was collected for rheological tests.

Small deformation oscillation measurements were carried out with a rheometer (TA Discovery HR-30), equipped with a Peltier plate, lower and upper flat plates (Cross-hatching pattern) with 40 mm diameter. All measurements were performed with a gap of 4 mm with a Peltier sensor at 25° C. The elastic modulus, G′, was obtained over a frequency range of 0.1-50 rad/sec and the strain was fixed at 0.1%. The hydrogels were subjected to a sweep frequency test with the rheometer and the value at an angular frequency of 10 rad/s was determined. The determination was made in triplicate. The reported G′ value is the average of the three determinations.

### Non-Clinical Safety Tests

The as-prepared superabsorbent polymer (Ex. 16 of Table 2) was weighed and infused into gelatin capsule to form a single use, ingestible, transiently space-occupying medical device. Referring to ISO 10993 “Biological evaluation of medical device”, the following biocompatibility and safety tests were assessed and cleared by accredited laboratories before human trial:

### In Vitro Cytotoxicity Test

L929 mouse fibroblast cells were obtained from ATCC (American Type Culture Collection, USA). 4 capsules of the SAP (containing a total of 2.24 g) were dissolved in 500 mL MEM and spread onto a 10 mm×10 mm filter membrane to form the SAP test sample. The negative control used was high density polyethylene from U.S. Pharmacopeial Convention (USP). The positive control used was natural latex gloves. Each of the control samples were prepared as 10 mm×10 mm samples.

Aseptic procedures were used for handling of cell cultures. L929 cells were cultured in Minimum Essential Media (MEM) medium (90% foetal bovine serum (FBS), Penicillin 100 U/mL, Streptomycin sulfate 100 μg/mL) at 37° C. in a humidified atmosphere of 5% CO₂ and then digested by 0.25% trypsin containing ethylenediaminetetraacetic acid (EDTA) to obtain a 1.0×10⁵ cells/mL suspension. The suspended cells were dispensed at 2 mL per vessel. Cell morphology was evaluated to verify that the monolayer was satisfactory after incubation at 37° C. in 5% CO₂ for 24 hours.

After the cells grew to form a monolayer, the original cell culture medium was discarded. Then 2 mL of fresh culture medium was added to each vessel. The SAP test sample was placed on the cell layer in the centre of each of the replicate vessels, ensuring that the SAP test sample covered approximately one tenth of the cell layer surface. The replicate vessels were prepared for both the negative control and positive control material in a similar manner. Three replicates of each group were tested.

After 48 hours of incubation, the outline of the test sample on the bottom of the culture dish was marked with a permanent marker, then the test sample was removed. The culture medium was aspirated and 500 μL of neutral red solution was added to each plate and incubated for 1 hour. The neutral red solution was poured off and 2 mL phosphate buffered saline (PBS) was added, then each culture was examined microscopically. Changes, for example in general morphology, vacuolization, detachment, cell lysis and membrane integrity was assessed using the criteria in Table A.

TABLE A
Reactivity grade

| Grade | Reactivity | Description of reactivity zone |
|---|---|---|
| 0 | None | No detectable zone around or under specimen |
| 1 | Slight | Some malformed or degenerated cells |
| | | under specimen |
| 2 | Mild | Zone limited to area under specimen |
| 3 | Moderate | Zone extending specimen size up to 10 mm |
| 4 | Severe | Zone extending farther than 10 mm beyond |
| | | specimen |

A numerical range greater than 2, based on Table A, was considered cytotoxic.

### Skin Sensitization Test

The SAP sample was extracted into 0.9% sodium chloride or sesame oil and the extract was evaluated to determine whether the components extracted from the SAP sample would cause skin sensitization in a guinea pig maximization test in accordance with ISO 10993-10:2010 “Part 10: Tests for irritation and skin sensitization”.

### 0.9% Sodium Chloride Injection Extract

The negative control was 0.9% Sodium Chloride Injection obtained from Guangxi Yuyuan Pharmaceutical Co., Ltd., and the positive control was 2,4-dinitrochlorobenzene (DNCB) obtained from Chengdu Aikeda Chemical Reagent Co., Ltd. 0.9% Sodium Chloride Injection is a 0.9% solution of sodium chloride in water.

Under aseptic conditions, samples were extracted using a whole sampling method, with additional volume of the extraction vehicle that the test sample absorbs when performing the extraction being added. The extraction was performed with agitation in closed inert containers according to the extraction ratio listed in Table B (sample: extraction vehicle). The extraction vehicle was 0.9% Sodium Chloride Injection.

TABLE B
Extraction using 0.9% Sodium Chloride Injection

| | | Extract Procedure | | | Final |
|---|---|---|---|---|---|
| Test Period | Actual Sampling | Extract Ratio | SC | Condition | Extract |
| Intradermal | 2.0 g | 0.2 g:1 mL | 210.0 mL | 50° C., 72 h | Not |
| Induction Phase I | | | | | Clear |
| Topical Induction | 2.0 g | 0.2 g:1 mL | 210.0 mL | 50° C., 72 h | Not |
| Phase II | | | | | Clear |
| Challenge Phase | 2.0 g | 0.2 g:1 mL | 210.0 mL | 50° C., 72 b | Not |
| | | | | | Clear |

The vehicle (without the SAP sample) was similarly prepared to serve as the control.

### Sesame Oil Extract

The negative control was sesame oil (SO) obtained from Ji'an Qingyuan District luyuanxiangliao. Co. Ltd., and the positive control was 2,4-dinitrochlorobenzene (DNCB) obtained from Chengdu Aikeda Chemical Reagent Co., Ltd.

Under aseptic conditions, samples were taken using a whole sampling method. The extraction was performed with agitation in closed inert containers according to the extraction ratio listed in Table C (sample: extraction vehicle). The extraction vehicle was sesame oil (SO).

TABLE C
Extraction using sesame oil

| | | Extract Procedure | | | |
|---|---|---|---|---|---|
| Test Period | Actual Sampling | Extract Ratio | SC | Condition | Final Extract |
| Intradermal | 2.0 g | 0.2 g:1 mL | 10.0 mL | 50° C., 72 h | Clear |
| Induction Phase I | | | | | |
| Topical Induction Phase II | 2.0 g | 0.2 g:1 mL | 10.0 mL | 50° C., 72 h. | Clear |
| Challenge Phase | 2.0 g | 0.2 g:1 mL | 10.0 mL | 50° C., 72 b | Clear |

The vehicle (without the SAP sample) was similarly prepared to serve as the control.

### Testing

Healthy male Hartley guinea pigs (Cavia porcellus) obtained from Suzhou Experimental Animal Sci-Tech Co., Ltd (Permit Code: SCXK (SU) 2020-0007) were used to evaluate skin sensitization. Initial body weight of each animal was 300 to 500 g. The animals were healthy and were not previously used in experimental procedures, and were kept in a bedding of corn cob (Suzhou shuangshi laboratory animal feed science Co. Ltd.) at a temperature of 18 to 26° C. at a humidity of 30% to 70% in a 12 hour light/dark cycle with full-spectrum lighting, and fed with a guinea pig diet (Suzhou Experimental Animal Sci-Tech Co., Ltd.).

For each of the experiments based on the 0.9% Sodium Chloride Injection extract or sesame oil extract, on the first day of treatment, 15 guinea pigs were weighed and identified. The fur from the dorsoscapular area of the animals was removed with an electric clipper, and the animals were grouped so that 10 animals were exposed to the extracts of the SAP sample and 5 animals were exposed to the negative control.

### I. Intradermal Induction Phase I

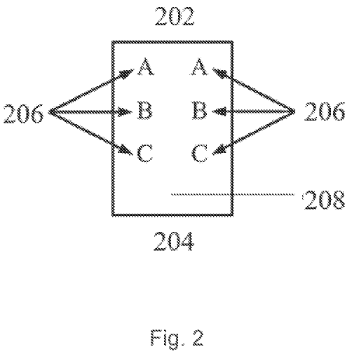

A pair of 0.1 mL intradermal injections were made into each animal at each of the injection sites (A, B and C) as shown in FIG. 2 in the clipped intrascapular region.
Site A: 50:50 (V/V) stable emulsion of Freund's complete adjuvant mixed with the chosen solvent.
Site B: the test sample (undiluted extract): the control animals were injected with solvent alone.
Site C: the test sample at the concentration used at site B, emulsified in a 50:50 (V/V) stable emulsion of Freund's complete adjuvant and the solvent; the control animals were injected with an emulsion of the blank liquid with adjuvant.

### II. Intradermal Induction Phase II

The maximum concentration that can be achieved in Intradermal induction phase I did not produce irritation. Animals were treated with 10% dodecyl sulfate (solvent: distilled water) 24±2 hours before the topical induction application.

At 7±1 days after completion of the intradermal induction phase, 0.5 mL of the SAP sample extract was administered by topical application to the intrascapular region of each animal, using a patch of area approximately 8 cm² (in an absorbent gauze), so as to cover the intradermal injection sites. The patches were secured with an occlusive dressing. The dressings and patches were removed after 48±2 hours. The control animals were treated similarly, using the blank liquid alone.

### III. Challenge Phase

At 14±1 days after completion of the topical induction phase, all test and control animals were challenged with the SAP sample. 0.5 mL of the test sample extract and control sample were administered by topical application to the sites that were not treated during the induction stage, using absorbent gauze (8 cm²) soaked in the SAP sample extract and control sample. The site was secured with occlusive dressing and the dressings and patches were removed after 24±2 hours.

The appearance of the challenged skin sites of the test and control animals were observed 24±2 hours and 48±2 hours after removal of the dressings. Full-spectrum lighting was used to visualize the skin reactions. The skin reactions for erythema and oedema were described and graded according to the Magnusson and Kligman grading.

### Oral Sensitization Test

The SAP sample was extracted into 0.9% sodium chloride or sesame oil and the extract was evaluated to determine whether the components extracted from the SAP sample would cause oral sensitization in hamsters in accordance with ISO 10993-10:2010 “Part 10: Tests for irritation and skin sensitization”.

0.9% Sodium Chloride Injection extract

The negative control was 0.9% Sodium Chloride Injection obtained from Guangxi Yuyuan Pharmaceutical Co., Ltd.

Under aseptic conditions, samples were taken using a whole sampling method, with additional volume of the extraction vehicle that the test sample absorbs when performing the extraction being added. The extraction was performed with agitation in closed inert containers according to the extraction ratio listed in Table D (sample: extraction vehicle). The extraction vehicle was 0.9% Sodium Chloride Injection.

TABLE D
Extraction using 0.9% Sodium Chloride Injection

| | | Extract Procedure | | | Final |
|---|---|---|---|---|---|
| Test Period | Actual Sampling | Extract Ratio | SC | Condition | Extract |
| Test extract | 2.0 g | 0.2 g:1 mL | 210.0 mL | 50° C., 72 h | Not |
| | | | | | Clear |
| Negative control | / | / | 5.0 mL | 50° C., 72 h | Clear |

The vehicle (without SAP test sample) was similarly prepared to serve as the control.

### Sesame Oil Extract

The negative control was sesame oil (SO) obtained from Ji'an Qingyuan District luyuanxiangliao. Co. Ltd.

Under aseptic conditions, samples were taken using a whole sampling method. The extraction was performed with agitation in closed inert containers according to the extraction ratio listed in Table E (sample: extraction vehicle). The extraction vehicle sesame oil (SO).

TABLE E
Extraction using sesame oil

| | | Extract Procedure | | | |
|---|---|---|---|---|---|
| Test Period | Actual Sampling | Extract Ratio | SC | Condition | Final Extract |
| Test extract | 2.0 g | 0.2 g:1 mL | 10.0 mL | 50° C., 72 h | Clear |
| Negative control | / | / | 5.0 mL | 50° C., 72 h | Clear |

The vehicle (without SAP test sample) was similarly prepared to serve as the control.

### Testing

Healthy male hamsters obtained from Beijing Vital River Laboratory Animal Technologies Co. Ltd. (Permit Code: SCXK (JING) 2016-0011) were used to evaluate oral sensitization. Initial body weight of each animal was 109 to 129 g. The animals were healthy and were not previously used in experimental procedures, and were kept in a bedding of corn cob (Suzhou shuangshi laboratory animal feed science Co. Ltd.) at a temperature of 18 to 26° C. at a humidity of 30% to 70% in 12 hour light/dark cycle with full-spectrum lighting, and fed with a irradiation sterilization feed (Suzhou shuangshi laboratory animal feed science Co. Ltd.).

For each of the experiments based on the 0.9% Sodium Chloride Injection extract or sesame oil extract, 6 animals were weighed and identified. The cheek pouches of the animals were inverted and washed with 0.9% Sodium Chloride Injection, and examined for any abnormalities. A cotton-wool pellet soaked in the SAP sample and placed in one pouch of each animal. No sample was placed in the other cheek pouch, which served as a control. The duration of exposure was 5 minutes. Following the exposure, the cotton-wool pellet was removed and the pouches were washed with 0.9% Sodium Chloride Injection, taking care not to contaminate the other pouch. The procedure was repeated every 1 hour for a duration of 4 hours. The control animals were treated similarly, using the negative control sample alone. The appearance of the cheek pouches of each animal was described and the pouch surface reactions were graded for erythema.

At 24±2 hours after the final treatment, the cheek pouches were examined macroscopically, and the hamsters were humanely sacrificed to remove the tissue samples from representative areas of the pouches. The tissue samples were placed in 4% formaldehyde prior to processing for histological examination. After fixation, the specimen were trimmed, embedded, sectioned and stained with a hematoxylin and eosin (H&E) stain. The irritant effects on the stained oral tissue was evaluated microscopically.

### Acute Systemic Toxicity Test

The SAP sample was extracted into 0.9% sodium chloride or sesame oil and the extract was evaluated to determine whether the components extracted from the SAP sample would cause acute systemic toxicity following injection into mice in accordance with ISO 10993-11:2017 “Part 11: Tests for systemic toxicity”.

### 0.9% Sodium Chloride Injection Extract

The negative control was 0.9% Sodium Chloride Injection obtained from Guangxi Yuyuan Pharmaceutical Co., Ltd.

Under aseptic conditions, samples were taken using a whole sampling method, with additional volume of the extraction vehicle that the test sample absorbs when performing the extraction being added. The extraction was performed with agitation in closed inert containers according to the extraction ratio listed in Table F (sample: extraction vehicle). The extraction vehicle was 0.9% Sodium Chloride Injection.

TABLE F
Extraction using 0.9% Sodium Chloride Injection

| | | Extract Procedure | | | Final |
|---|---|---|---|---|---|
| Test Period | Actual Sampling | Extract Ratio | SC | Condition | Extract |
| Test extract | 2.0 g | 0.2 g:1 mL | 210.0 mL | 50° C. , 72 h | Not |
| | | | | | Clear |
| Negative control | / | / | 5.0 mL | 50° C., 72 h | Clear |

The vehicle (without SAP test sample) was similarly prepared to serve as the control.

### Sesame Oil Extract

The negative control was sesame oil (SO) obtained from Ji'an Qingyuan District luyuanxiangliao. Co. Ltd.

Under aseptic conditions, samples were taken using a whole sampling method. The extraction was performed with agitation in closed inert containers according to the extraction ratio listed in Table G (sample: extraction vehicle). The extraction vehicle sesame oil (SO).

TABLE G
Extraction using sesame oil

| | | Extract Procedure | | | |
|---|---|---|---|---|---|
| Test Period | Actual Sampling | Extract Ratio | SC | Condition | Final Extract |
| Test extract | 2.0 g | 0.2 g:1 mL | 10.0 mL | 50° C., 72 h | Clear |
| Negative control | / | / | 5.0 mL | 50° C., 72 h | Clear |

The vehicle (without SAP test sample) was similarly prepared to serve as the control.

### Testing

Healthy male ICR mice obtained from Zhejiang Vital River Laboratory Animal Technology Co. Ltd., (Permit Code: SCXK (Zhe) 2019-0001) were used to evaluate acute systemic toxicity. Initial body weight of each animal was 18 to 22 g. The animals were healthy, young and were not previously used in experimental procedures, and were kept in a bedding of corn cob (Suzhou shuangshi laboratory animal feed science Co. Ltd.) at a temperature of 20 to 26° C. at a humidity of 30% to 70% in 12 hour light/dark cycle with full-spectrum lighting, and fed with a guinea pig diet (Suzhou Experimental Animal Sci-Tech Co., Ltd.).

For each of the experiments based on the 0.9% Sodium Chloride Injection extract or sesame oil extract, on the first day of treatment, 10 mice were weighed and identified and grouped so that 5 animals were exposed to the SAP sample and 5 animals were exposed to the negative control. A single dose of the test sample extract was administered to the designated group of mice by oral gavage at a dosage of 50 mL/kg. The negative control was administered similarly to the control group. After administration of the sample, food was withheld for an additional 3 hours to 4 hours.

The mice were observed for any adverse clinical reactions immediately after injection, and the animals were returned to their cages. The animals were observed for signs of systemic reactions at 4, 24, 48 and 72 hours post administration and weighed daily for three days after administration. Any animal found dead or abnormal sigs were subjected to gross necroscopy.

If during the observation period of an acute systemic toxicity test, none of the mice treated with the test article extract exhibited a significantly greater biological reactivity than the control mice, the SAP test sample was considered to have met the requirements of no acute systemic toxicity. If two or more animals died, or if abnormal behaviour such as convulsions or prostration occurred in two or more animals, or if body weight loss greater than 10% occurred in three or more animals, the SAP sample was considered not to have met the requirements and were considered to have acute systemic toxicity.

### Example 1: Synthesis

### Synthesis of Spacer Crosslinkers With Catalysts (Step 1, FIG. 1 (101))

Citric acid (CA, 1 g) and catalysts (SHP or CAT2, 0.5 g) were dissolved into 10 mL DI water. PEG with different lengths were weighed and gradually added to the CA solution. The fully dissolved solution was charged in a flask of a rotary evaporator (IKA) with a silicone oil bath. The solution in the rotary flask was heated at 100° C. for 0.5 hours, then the oil bath temperature was increased to 120° C. gradually. Without condensation, all the water in the flask evaporated after 2 hours, resulting in a viscous yellow coloured paste in the flask. Once cooled down to room temperature (RT), the resultant paste was further diluted with DI water to form a 30 mL sample, from which 1.5 or 3 mL (equivalent to 50 or 100 mg CA, respectively) was used for further crosslinking reaction or titration.

### Synthesis of Spacer Crosslinkers Without Catalysts (Step 1, FIG. 1 (101))

Citric acid (CA, 1 g) was dissolved into 10 mL DI water, then PEG with different lengths were weighted and mixed with CA solution. Fully dissolved solution was charged in a flask of a rotary evaporator (IKA) with a silicone oil bath. The solution in the rotary flask was heated at 100° C. for 0.5 hours, then the oil bath temperature was increased to 120° C. gradually. Without condensation, all the water in the flask evaporated after 2 hours, resulting in a viscous yellow coloured paste in the flask. Once cooled down to RT, the resultant viscous paste was dissolved in DI water to form a 30 mL solution, from which 1.5 or 3 mL (equivalent to 50 or 100 mg CA, respectively) was used for further crosslinking reaction and titration.

Preparation of Crosslinked Carboxymethylcellulose Hydrogels without Polymer Additive (Step 2, FIG. 1 (102))

DI water (400-700 mL) was added to a 1 L beaker and stirred with ANGNI electric mixer at 60 rpm. Solutions of the spacer crosslinkers with equivalent citric acid content (equivalent to 50 mg or 25 mg CA) was added to the water. CMC (10 g) was then added to the solution and the resulting mixture was agitated at room temperature at 120 rpm for 2 hours and then at 60 rpm for 24 hours. The final homogenised solution was poured into a stainless steel tray with a solution thickness of less than 2 cm. The tray was placed in a convection oven (Lantian) at 50° C. for 24 hours. The tray was removed from the oven, and the dried CMC sheet was inverted and the tray was placed back in the oven and maintained at 50° C. for 12 to 24 hours until no change in weight was observed.

After full desiccation, the CMC sheet was ground by means of a cutting blender (Philips). The granulated material was sieved to a particle size of from 0.1 mm to 2 mm, then spread on the tray and crosslinked in the convection oven (Binder) at 120° C. for 2 to 4 hours. The crosslinked polymer hydrogel thus obtained was washed with DI water over 4 to 12 hours by changing the washing solution 3 times to remove unreacted reagents. The washing stage increased the hydrogel's media uptake capacity by increasing the relaxation of the network. After washing, the hydrogel was placed on a tray and placed in an oven (Lantian) at 50° C. for 12 to 24 hours until no change in weight was observed. The dried hydrogel aggregates were ground and sieved to a particle size from 0.1 mm to 1 mm. The experiments described below were performed using the inventive polymer as is (without further processing), unless otherwise specified. However, the inventive polymer can be infused in gelatin capsules and sealed for further biomedical studies.

Preparation of Crosslinked Carboxymethylcellulose Hydrogels with Polymer Additive

To prepare the crosslinked carboxymethylcellulose hydrogels with the polymer additive, a similar procedure as the preparation of crosslinked carboxymethylcellulose hydrogels without polymer additive was used, except PEG oligomer (100-300 mg) was added to the water together with the spacer crosslinkers and citric acid, before the addition of the CMC. All subsequent procedures were repeated in the same manner.

### Large Scale Preparation of Crosslinked Carboxymethylcellulose Hydrogels

A larger scale preparation of the crosslinked carboxylmethylcelullose hydrogel with polymer additive was performed as follows:

Citric acid (CA, 5 g) was dissolved into 50 mL of DI water. PEG4000 (50 g) was then added into the CA solution. The fully dissolved solution was charged in a flask of a rotary evaporator and heated at 98° C. for 8 hours and the resultant paste was fully dissolved in DI water to form a 200 mL spacer crosslinker solution.

DI water (6 L) was added to a 10 L container and stirred at 60 rpm. The spacer crosslinker solution (20 mL), CMC (100 g) and PEG200 (1 g) was added and the resulting mixture was agitated at room temperature at 100 rpm for 24 hours to obtain a homogenised solution. The homogenised solution was poured into stainless steel trays and the trays were placed in a convection oven at 80° C. for 24 hours to obtain dried composite sheets. The dried composite sheets were then mechanically grounded and sieved to a sieved particle with a size of around 1.0 mm The sieved particles were then heated at 100° C. for 8 hours to obtain a crosslinked hydrogel.

### Example 2: Analysis of Spacer Crosslinkers

Table 1 shows the titration summary of the spacer crosslinkers after the Step 1 reaction (FIG. 1, (101)) and corresponding mixture controls, where the equivalent amounts of the components were simply mixed without covalent bonding. Specifically, PEG-CA represents the spacer crosslinker where PEG and CA are covalently bonded, while PEG+CA represents the mixture control where PEG and CA are simply mixed. The number indicated after PEG (200, 400, 1000, 2000) indicates the molecular weight of the PEG.

Successful esterification between PEG hydroxyl groups and CA carboxylic acid groups were shown by the significantly reduced volume of base required during titration, in comparison with the mixture control where PEG and CA were simply mixed. Taking PEG200-CA without catalysts and PEG200+CA (mixture control) as an example, it can be seen that the mixture control consumed 17.2 mL 0.1N NaOH which was almost the same as pure CA control without PEG200. In contrast, after the Step 1 reaction (FIG. 1, (101)), the volume of 0.1N NaOH required for titration dropped to 12.0 mL. Using Calculation Method A (indicated above under characterization method of esterification between PEG with CA), the estimated esterification degree for PEG200-CA was found to be about 94%.

A similar comparison may be made with the Step 1 reaction (FIG. 1, (101)) using catalysts. “:SHP” or “:CAT2” indicates that the spacer crosslinker was formed in the presence of the respective catalyst. For PEG200-CA with SHP as catalyst (PEG200-CA:SHP) and PEG400-CA with SHP as catalyst (PEG400-CA:SHP), the degree of esterification was found to be approximately 84 and 87%, respectively.

There was no obvious evidence to justify the efficiency of using a catalyst for the esterification process over not using a catalyst. For example, the degree of esterification of PEG400-CA with and without SHP are 86.3% and 98.7%, respectively. It is known that SHP can weaken the hydrogen bonding between CA carboxylic acid groups, contributing to accelerated anhydride formation at low temperature. SHP also accelerates the formation of anhydride intermediates by polycarboxylic acid in an amorphous state. Dual catalysts CAT2 have been implicated in cellulose or PEG esterification in the past, but there have been difficulties in quantifying the Step 1 reaction (FIG. 1, (101)), because sodium bicarbonate in CAT2 immediately reacts with CA to generate carbon dioxide bubbles. The mixture control of PEG200+CA+CAT2 only consumed 13.2 mL NaOH. The other component in CAT2, disodium phosphate, can form McIlvaine buffer with CA and significantly disturb the real neutral point during the base titration. Therefore, if Calculation Method A (indicated above under characterization method of esterification between PEG with CA) is applied to PEG200-CA:CAT2, an artefactual degree of esterification of over 100% is observed, as indicated with (*) in Table 1.

TABLE 1
Base titration summary of spacer crosslinkers and corresponding mixture control

| | NaOH | | | |
|---|---|---|---|---|
| Reagents/ | (mL)/CA | [COOH] | | |
| Composition | (100 mg) | Reduction % | Esterification Degree (%) | Remark |
| CA | 17.2 | / | / | Control |
| PEG200 + CA + SHP | 17.0 | / | / | Mixture Control |
| PEG200 − CA:SHP | 12.4 | 27.1 | 84.7 | Step 1 Reacted |
| PEG200 + CA + CAT2 | 13.2 | / | / | Mixture Control |
| PEG200 − CA:CAT2 | 6.8 | 48.5 | 152%* | Step 1 Reacted |
| PEG400 + CA + SHP | 17.0 | / | / | Mixture Control |
| PEG400 − CA:SHP | 12.3 | 27.6 | 86.3 | Step 1 Reacted |
| PEG200 + CA | 17.2 | / | / | Mixture Control |
| PEG200 − CA | 12.0 | 30.2 | 94.4 | Step 1 Reacted |
| PEG400 + CA | 17.1 | / | / | Mixture Control |
| PEG400 − CA | 11.7 | 31.6 | 98.7 | Step 1 Reacted |
| PEG1000 + CA | 17.5 | / | / | Mixture Control |
| PEG1000 − CA | 13.0 | 25.7 | 80.3 | Step 1 Reacted |
| PEG2000 + CA | 17.0 | / | / | Mixture Control |
| PEG2000 − CA | 11.6 | 31.7 | 99.2 | Step 1 Reacted |
| PEG4000 + CA | 16.8 | / | / | Mixture Control |
| PEG4000 − CA | 12.4 | 26.2 | 81.9 | Step 1 Reacted |

*indicates a degree of esterification of greater than 100%, which is an experimental/calculation artefact.

### Example 3: Analysis of CMC Hydrogels with Spacer Crosslinkers

The obtained PEG-CA spacer solutions with or without catalysts were directly used for Step 2 CMC crosslinking process (FIG. 1, (102)). No further purification was carried out because a small amount of unreacted CA and PEG can take part in the following crosslinking process at high temperature, and the unreacted catalysts can be removed during the hydrogel washing process. Table 2 summaries the properties of the crosslinked CMC (X-CMC) hydrogels with spacer crosslinkers under various conditions, including using equivalent CA/CMC wt %, PEG-CA crosslinkers with different PEG chain length, crosslinkers with or without catalysts, crosslinking temperature and timing. Two of most important parameters, water absorbance (MUR) and mechanical strength (G′), were measured to evaluate the performance of the hydrogels. From Table 2, several critical conclusions on the design and fabrication of the hydrogel could be drawn.

### Effect of Equivalent CA/CMC wt % on Hydrogel MUR

In the Step 2 reaction (FIG. 1, (102)), the CA/CMC wt % ratio was adjusted by adding different volumes of spacer crosslinker solutions (for example, 1.5 mL PEG-CA equivalent to 50 mg CA) into a solution of a fixed amount of CMC (10 g), then proceeding with the drying and crosslinking processes to obtain the hydrogels. A significant trend was observed when the equivalent CA wt % in the spacer crosslinker solution was reduced from 1% to 0.25%, whereby the MUR of the hydrogel was found to increase from <30 to >90. A similar trend has been previously observed when pristine CA was used as the crosslinker. A lower CA wt % means that less crosslinking reaction occurs between CA carboxylic acid groups and the CMC hydroxyl groups, resulting in lower degree of crosslinking and a looser polymeric network. For hydrogels, a looser polymeric network typically results in greater water absorbance or swelling ratio. Even though the molecular size of the inventive PEG-CA crosslinkers are much larger than CA molecules, the crosslinking reaction still proceeds between the carboxylic acid end groups of PEG-CA and the hydroxyl groups of the CMC backbone. A more accurate estimation on the degree of CMC crosslinking can be ascertained from the free carboxylic acid groups present on the ends of PEG-CA crosslinkers, since about a third (⅓) of the carboxylic acid groups of the CA is consumed during the Step 1 esterification process (FIG. 1, (101)). For example, a molecule of pristine CA has three COOH groups and two molecules of CA has six COOH groups. After esterification, one PEG-CA crosslinker comprises PEG covalently bonded to two CA at each end, and each PEG-CA crosslinker will only have four free COOH groups, since the third COOH group on each CA molecule would have reacted to form a covalent bond with the PEG. In this regard, Table 1 also shows the titrated NaOH volume of the respective mixture controls which is an indication of the amount of free COOH groups available for the crosslinking process.

### Effect of Catalyst on Hydrogel MUR

Similarly to the Step 1 esterification (FIG. 1, (101)) results, there was no clear evidence to show that the incorporated catalyst in the crosslinker solution contributed to any advantageous hydrogel properties. Taking Ex. 4 and Ex. 7 of Table 2 for comparison, the MUR of PEG200-CA (equivalent to 50 mg CA) crosslinked CMC with and without SHP are similar. The same could be said for PEG400-CA crosslinked CMC hydrogels (Ex. 6 and Ex. 8 of Table 2). From an economic and commercial perspective, this shows that a catalyst is not necessary for large scale manufacturing of the hydrogel.

It is worth mentioning the comparison between SHP and CAT2 catalysed examples, for example Ex. 2 and Ex. 3, or Ex. 4 and Ex. 5 of Table 2. Even though the initial PEG-CA crosslinkers used in these examples had the same equivalent CA wt %, the absorbance capability of the resulting hydrogels were quite different. Specifically, Ex. 2 of Table 2 with SHP had a MUR of about 24, while Ex. 3 of Table 2 with CAT2 had a MUR of about 52. This can be explained by the titrated NaOH volume in the crosslinker solution. Due to the sodium bicarbonate and phosphate buffer effects as mentioned above, less COOH groups remained in CAT2 when used in the PEG-CA crosslinker reactions, leading to a lower crosslinking density in the resulting hydrogel.

### Effect of PEG on Hydrogel MUR and G′

When long hydrophilic crosslinkers such as PEG is used, one end of the crosslinker reacts with the CMC chain, and due to the flexible nature of the polymer chain in the crosslinker, the other end can move around and have a higher chance of reacting with another CMC chain located further away. This overcomes the issues of low mobility when using a short crosslinker like CA. Therefore, robust yet loose hydrogel networks achieved using long hydrophilic crosslinkers tend to have higher elastic modulus coupled with higher water absorbance.

This feature was well verified by the examples in Table 2. Ex. 11 to 14 using spacer crosslinkers with the same equivalent CA/CMC ratio demonstrated significantly higher MUR and G′ compared to C-1 control hydrogel crosslinked with CA.

Further, it was observed that spacer crosslinkers with higher molecular weight or longer chain length results in hydrogels with higher MUR: Ex. 14 of Table 2 with PEG2000 was shown to have a MUR of about 140, while Ex. 11 of Table 2 with PEG200 was shown to have a MUR of about 90. As mentioned above, using a longer hydrophilic crosslinker will lead to a looser polymeric network, thus a higher water absorbance. However, the correlation between crosslinker PEG length and the elastic modulus G′ of the hydrogel is not linear: Ex. 12 of Table 2 with PEG400 was shown to have a G′ of about 2300 while Ex. 14 of Table 2 with PEG2000 had a G′ of about 1600. From a comparison of the MUR values of Ex. 11 to 14 of Table 2, it can be seen that the mechanical strength of the hydrogel is more sensitive and is oppositely correlated with water absorbance.

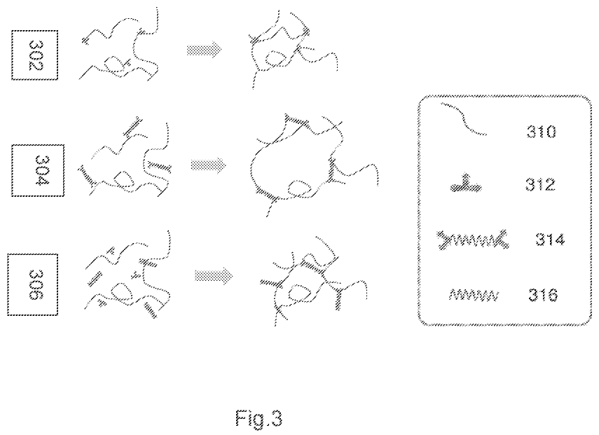

It should also be noted that for the simple mixture of PEG2000+CMC+CA (control C-2), the MUR was about 80 and G′ was about 1300. A schematic drawing of the crosslinking mechanisms as shown in FIG. 3 also explains the difference in properties. For control C-2, random esterification occurred between the hydroxyl groups of CMC/PEG and the carboxylic acid groups of CA molecules. Considering that the CMC polymer backbone contains a significantly larger amount of pendant hydroxyl groups compared to the end hydroxyl groups of PEG, most of the esterification reaction in the control C-2 took place between CMC and CA, resulting in a similar crosslinking effect to that of control C-1. In control C-2, only a small amount of the PEG would have been crosslinked into the CMC network, which would change the rheological property of the hydrogel.

TABLE 2
Summary of crosslinked CMC with spacer crosslinkers and CA controls

| | | Eq. CA | NaOH | | |
|---|---|---|---|---|---|
| Ex. # | Composition | (mg)/CMC (10 g) | (mL)/Eq. CA (mg) | MUR | G′ (Pa) |
| 1 | X-CMC/PEG200 − CA:SHP | 100 (1%) | 12.4 | 16.8 | 4803 |
| 2 | X-CMC/PEG200 − CA:SHP | 100 (1%) | 12.4 | 24.2 | 4266 |
| 3 | X-CMC/PEG200 − CA:CAT2 | 100 (1%) | 6.8 | 51.6 | 2908 |
| 4 | X-CMC/PEG200 − CA:SHP | 50 (0.5%) | 6.2 | 58.4 | 2794 |
| 5 | X-CMC/PEG200 − CA:CAT2 | 50 (0.5%) | 3.4 | 74.8 | 2370 |
| 6 | X-CMC/PEG400 − CA:SHP | 50 (0.5%) | 6.2 | 67.4 | 2885 |
| 7 | X-CMC/PEG200 − CA | 50 (0.5%) | 6.0 | 53.8 | 2986 |
| 8 | X-CMC/PEG400 − CA | 50 (0.5%) | 5.8 | 61.2 | 3015 |
| 9 | X-CMC/PEG1000 − CA | 50 (0.5%) | 6.5 | 57.8 | 3223 |
| 10 | X-CMC/PEG2000 − CA | 50 (0.5%) | 5.8 | 66.8 | 3029 |
| 11 | X-CMC/PEG200 − CA | 25 (0.25%) | 3.0 | 92.0 | 2014 |
| 12 | X-CMC/PEG400 − CA | 25 (0.25%) | 2.9 | 96.6 | 2289 |
| 13 | X-CMC/PEG1000 − CA | 25 (0.25%) | 3.2 | 104.4 | 2072 |
| 14 | X-CMC/PEG2000 − CA | 25 (0.25%) | 2.9 | 140.8 | 1568 |
| C-1 | X-CMC/CA | 25 (0.25%) | 4.3 | 82.8 | 1260 |
| C-2 | X-CMC/PEG2000 + CA | 25 (0.25%) | 4.2 | 77.2 | 1291 |
| 15 | X-CMC/PEG4000 − CA | 25 (0.25%) | 3.1 | 142.6 | 1637 |
| 16 | X-CMC/PEG4000 − CA/PEG200 | 25 (0.25%) | 3.1 | 146.6 | 2164 |

### Example 4: Non-Clinical Safety Tests

As prepared superabsorbent polymer (Ex. 16 of Table 2) was weighed and infused into gelatin capsules to form a single use, ingestible, transiently space-occupying medical device. It was classified as a mucosal membrane contacting device as it involved repeat, prolonged contact during use (>24 hours, <30 days). The following biocompatibility and safety tests were assessed and cleared by accredited laboratories before human trial:

### In Vitro Cytotoxicity

In vitro cytotoxicity was evaluated using a mammalian cell culture (L929) direct contact method in accordance with ISO 10993-5:2009 “Part 5: Tests for in vitro cytotoxicity”.

The results are shown in Table 3 and Table 4.

TABLE 3
Observation of Cell Morphology

| | After | Before | |
|---|---|---|---|
| Group | inoculation | treated with test article | 48h after treatment |
| Test article | Discrete | Discrete | Zone limited to area under specimen |
| Negative | intracytoplasmic | intracytoplasmic | No detectable zone around or under |
| control | granules; no cell | granules; no cell | specimen. |
| Positive | lysis,no reduction | lysis,no reduction | Zone extending farther than 10 mm |
| control | of cell growth | of cell growth | beyond specimen. |

TABLE 4
Cell Reactivity

| Group | Parallel 1 | Parallel 2 | Parallel 3 | Average | Interpretation |
|---|---|---|---|---|---|
| Test | 2 | 2 | 2 | 2 | Mildly |
| Group | | | | | cytotoxic |
| Negative | 0 | 0 | 0 | 0 | None cytotoxic |
| Control | | | | | |
| Positive | 4 | 4 | 4 | 4 | Severely |
| Control | | | | | cytotoxic |

Under the conditions tested, the SAP sample did not show potential toxicity to L929 cells.

### Skin Sensitization

Skin sensitization tests (0.9% NaCl and Sesame Oil extracts) were performed using a guinea pig maximization test in accordance with ISO 10993-10:2010 “Part 10: Tests for irritation and skin sensitization”.

### 0.9% Sodium Chloride Injection Extract

No skin sensitization reaction was found in the skin of guinea pigs using extracts of the SAP sample, and the positive rate of sensitization was 0%. The positive rate of sensitization in the positive control group was 100%.

### Sesame Oil Extract

No skin sensitization reaction was found in the skin of guinea pigs using extracts of the SAP sample, and the positive rate of sensitization was 0%. The positive rate of sensitization in the positive control group was 100%.

### Oral Mucosa Irritation

Oral mucosa irritation tests (0.9% NaCl and Sesame Oil extracts) were performed on hamsters in accordance with ISO 10993-10:2010 “Part 10: Tests for irritation and skin sensitization”.

### 0.9% Sodium Chloride Injection Extract

Under the conditions of the experiment, the SAP sample did not show any significant evidence of causing oral mucosa irritation in hamsters.

Microscopic histopathological evaluation showed that in the oral mucosa structure of the test group and control group, the stratified squamous epithelium and lamina propria, were in normal condition. In the stratified squamous epithelium, each layer of cells was normal and intact, and leucocyte infiltration, vascular congestion and oedema was not observed. The lamina propria of the test group and control group were normal and intact, and leucocyte infiltration, vascular congestion and oedema were not observed. In the lamina propria of the test group and control group, there was no oedema in the small blood vessel wall, part of the tube concretions were observed within a few red blood cells, and leucocyte infiltration was not observed in the surrounding vessels. The salivary glands could be seen in the lamina propria of the test group and the control group, and the structure of the salivary glands was normal and intact, with no enlargement of acinus, and leucocyte infiltration and oedema was not observed around the acinus. No deformation, leucocyte infiltration or oedema was observed in the skeletal muscle fibre under the oral mucosa of the test group and control group.

### Sesame Oil Extract

Under the conditions of the experiment, the SAP sample did not show any significant evidence of causing oral mucosa irritation in hamsters.

Microscopic histopathological evaluation showed that in the oral mucosa structure of the test group and control group, the stratified squamous epithelium and lamina propria, were in normal condition. In the stratified squamous epithelium, each layer of cells was normal and intact, and leucocyte infiltration, vascular congestion and oedema was not observed. The lamina propria of the test group and control group were normal and intact, and leucocyte infiltration, vascular congestion and oedema were not observed. In the lamina propria of the test group and control group, there was no oedema in the small blood vessel wall, part of the tube concretions were observed within a few red blood cells, and leucocyte infiltration was not observed in the surrounding vessels. The salivary glands could be seen in the lamina propria of the test group and the control group, and the structure of the salivary glands was normal and intact, with no enlargement of acinus, and leucocyte infiltration and oedema was not observed around the acinus. No deformation, leucocyte infiltration or oedema was observed in the skeletal muscle fibre under the oral mucosa of the test group and control group.

### Acute Systemic Toxicity

Acute systemic toxicity tests (0.9% NaCl and Sesame Oil extracts) were performed on mice by oral administration/gavage, in accordance with ISO 10993-11:2017 “Part 11: Tests for systemic toxicity”.

### 0.9% Sodium Chloride Injection Extract

All animals appeared clinically normal throughout the study. Body weight data were acceptable and equivalent between the test and control treatment groups.

### Sesame Oil Extract

All animals appeared clinically normal throughout the study. Body weight data were acceptable and equivalent between the test and control treatment groups.

### Example 5: Human Volunteer Study

To verify the efficiency of the superabsorbent polymer hydrogels (SAPs) on the treatment of excessive weight and obesity, a capsule device comprising SAP Ex.16 of Table 2 was tested using two middle aged healthy but overweight female volunteers having a BMI of about 28, whereby one was administered SAP, while the other was administered a placebo. The volunteers were placed on a normal average mixed diet and monitored over 12 weeks.

For administration, volunteer I consumed 500 mL of water with 4 capsules (containing a total of 2.24 g of SAP Ex.16 of Table 2) and volunteer II consumed 500 mL of water with 4 capsules (containing a total of 2.24 g of food grade sugar) at least 30 minutes before each meal. Both volunteers were prescribed a hypocaloric diet of 300 kcal per day below their calculated energy requirement and were instructed to perform daily moderate-intensity exercise such as 30 minutes of walking per day during the study.

As shown in Table 5, a significant body weight change was observed for volunteer I compared to volunteer II (6.3% and 2.0%, respectively) after 12 weeks, despite their similar initial body mass index (BMI) of about 28. The significant increase in weight loss in volunteer I is attributed to the SAP hydrogels which functioned as a gastric space-occupying device and helped volunteer I easily control food intake. Proven by volunteer II, healthy lifestyle like diet control and exercise did help somewhat but additional measures were needed to boost the weight loss effect to achieve a well-accepted −5% response ratio.

During the study, the frequency of the volunteers' bowel motion and life quality score were recorded to explore the effect of the SAP on functional constipation. Chronic constipation is a common disorder characterized by infrequent bowel movements, hard stools, and difficulty passing stool. Constipation has traditionally been treated with fibres, osmotic agents, and stimulants, such as psyllium, polyethylene glycol, and bisacodyl, respectively.

As shown in Table 5, volunteer I experienced more frequent and regular bowel movement after being administered with the SAP capsules. Quality of life was measured referring to modified SF-36 health survey and the Impact of Weight on Quality of Life-Lite. The modified SF-36 evaluated 8 domains (physical function, role physical, bodily pain, general health, vitality, social function, role emotional, mental health), and the scores ranged from 0 (poorest health status) to 10 (best health status). Remarks in the surveys showed that there was much less constipation symptoms for volunteer I compared to volunteer II during the study period, which corresponded with their life quality scores. The scientific explanation of the function of the SAP in constipation is due to its water storage and retention capability. The SAP hydrogels may be partially degraded by the bacteria in the colon, therefore may release water and cellulose fibre which help to ameliorate the constipation.

TABLE 5
Comparison of the effect of SAP on a human
subject with control
Human research

| Volunteer | I (Ex.16) | II (Sugar) |
|---|---|---|
| Body weight change (%) | | |
| W1 | 0.0 | 0.0 |
| W2 | −0.8 | −0.5 |
| W3 | −1.3 | −0.5 |
| W4 | −2.3 | −0.8 |
| W5 | −3.5 | −1.4 |
| W6 | −3.5 | −1.1 |
| W7 | −3.9 | −0.5 |
| W8 | −4.3 | −1.5 |
| W9 | −5.0 | −1.8 |
| W10 | −5.2 | −1.2 |
| W11 | −6.1 | −1.7 |
| W12 | −6.3 | −2.0 |
| Numbers of pass motion | | |
| W1 | 5 | 7 |
| W6 | 7 | 6 |
| W12 | 7 | 6 |
| Life quality score (1-10) | | |
| W1 | 6.3 | 7.4 |
| W6 | 7.9 | 7.1 |
| W12 | 8.2 | 7.3 |

### INDUSTRIAL APPLICABILITY

This invention may be used in personal disposable hygiene products, such as baby diapers, adult diapers and sanitary napkins, blocking water penetration in underground power or communications cable, in self-healing concrete, horticultural water retention agents, control of spill and waste aqueous fluid, and artificial snow for motion picture and stage production.

This invention may also be used in the treatment of obesity, pre-diabetes, diabetes, non-alcoholic fatty liver diseases, chronic idiopathic constipation, and in reducing caloric intake or improving glycemic control. This invention may also be used in a method of weight-loss or improving the body appearance in a healthy subject.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A polymer comprising a polysaccharide crosslinked with a spacer crosslinker, wherein the spacer crosslinker comprises a first optionally substituted aliphatic moiety terminated at each end with a second moiety comprising at least two carboxylic acid groups.

2. The polymer according to claim 1, wherein the spacer crosslinker has the following formula (I):
A-L-Z-L-A (I)
wherein
Z is the first optionally substituted aliphatic moiety;
A is the second moiety comprising at least two carboxylic acid groups; and
L is a linking group.

3. The polymer according to claim 1, wherein the first optionally substituted aliphatic moiety is derived from a first optionally substituted aliphatic molecule comprising at least two hydroxy groups.

4. The polymer according to claim 3, wherein the first optionally substituted aliphatic molecule has a molecular weight in the range of about 0.1 kDa to about 100 kDa.

5. The polymer according to claim 3, wherein the first optionally substituted aliphatic molecule is a hydrophilic polymer, preferably selected from the group consisting of polyether, polyacrylamide, polyethyleneimine, polyacrylate, polymethacrylate, polyvinyl pyrrolidone and polyvinyl alcohol, each further comprising at least two hydroxy groups.

6. The polymer according to claim 1, wherein the first optionally substituted aliphatic moiety has the following structure 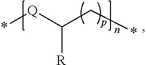
wherein Q is —CH₂—, —O— or —NH₂—,
R is hydrogen, —OH, optionally substituted C₁ to C₆ alkyl, —C(O)OM, —C(O)NR²R³ or optionally substituted heterocycloalkyl,
R² and R³ are independently hydrogen or optionally substituted C₁ to C₆ alkyl,
M is R², Na or K, p in an integer in the range of 1 to 6,
n is an integer in the range of 2 to 2000 and
* indicates where the moiety attaches to the rest of the spacer crosslinker.

7. The polymer according to claim 1, wherein the second moiety comprising at least two carboxylic acid groups is derived from a second molecule having at least three carboxylic acid groups.

8. The polymer according to claim 7, wherein the second molecule having at least three carboxylic acid groups is selected from the group consisting of citric acid, pyromellitic acid, butanetetracarboxylic acid, and benzoquinonetetracarboxylic acid.

9. The polymer according to claim 1, wherein the second moiety comprising at least two carboxylic acid groups is selected from the group consisting of: 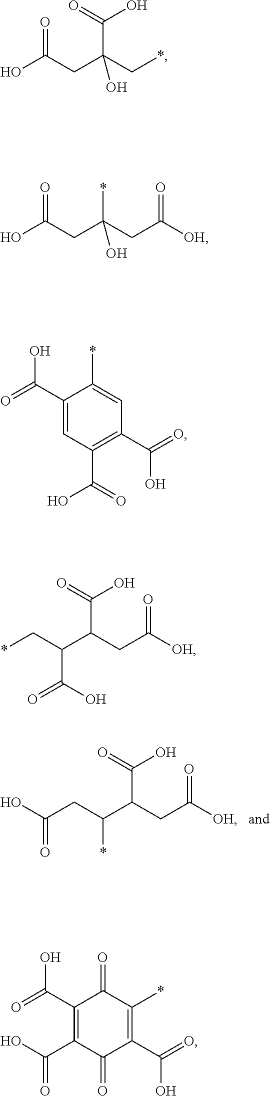
wherein * indicates where the moiety attaches to the rest of the spacer crosslinker.

10. The polymer according to claim 2, wherein L is selected from the group consisting of an amide, ester, acid anhydride and thioester.

11. The polymer according to claim 1, wherein the polysaccharide is selected from the group consisting of starch, cellulose, galactomannan and alginate, or is carboxymethylcellulose.

12. The polymer according to claim 1, wherein the polymer is in the form of a powder having a particle size in the range of about 0.05 mm to about 5 mm.

13. A hydrogel comprising the polymer according to claim 1 and a liquid.

14. A method of forming the polymer according to claim 1, comprising steps of:
a) reacting a first optionally substituted aliphatic molecule comprising at least two hydroxyl groups with a second molecule comprising at least three carboxylic acid groups to form a spacer crosslinker; and
b) crosslinking the spacer crosslinker with a polysaccharide to form the polymer of claim 1.

15. The method according to claim 14, wherein the reacting step (a) further comprises a polymer additive.

16. The method according to claim 14, wherein the reacting step (a) and crosslinking step (b) are independently performed at a temperature in the range of 80° C. to 180° C.

17. The method according to claim 15, further comprising the following steps (a1), (a2) and (a3) between the reacting step (a) and the crosslinking step (b):
a1) mixing the spacer crosslinker and the polysaccharide in a solvent to form a homogenized mixture,
a2) drying the homogenized mixture at a temperature in the range of 40° C. to 90° C. to remove the solvent, and
a3) grinding the dried homogenized mixture to form a powder having a particle size in the range of about 0.05 mm to about 5 mm.

18. The method according to claim 17, wherein the mixing step (a1) further comprises the polymer additive.

19. The method according to claim 14, further comprising a step of adding a liquid to the polymer.

20. A composition comprising the polymer according to claim 1 and a pharmaceutically acceptable excipient.

21. The composition according to claim 20, further comprising a polymer additive.

22. A capsule comprising the polymer according to claim 1.

23. A method of treating obesity, pre-diabetes, diabetes, non-alcoholic fatty liver disease (NAFLD), or chronic idiopathic constipation, or of reducing caloric intake or improving glycemic control in a subject in need thereof, comprising a step of orally administering to subject a therapeutically effective amount of the polymer according to claim 1.

24. A use, comprising using the polymer according to claim 1 in a treatment of obesity, pre-diabetes, diabetes, non-alcoholic fatty liver disease (NAFLD), or chronic idiopathic constipation, or for reducing caloric intake or improving glycemic control.

25. A use, comprising using the polymer according to claim 1 in the manufacture of a medicament for a treatment of obesity, pre-diabetes, diabetes, non-alcoholic fatty liver disease (NAFLD), or chronic idiopathic constipation, or for reducing caloric intake or improving glycemic control.

26. The method of claim 23, wherein the polymer is administered or is to be administered orally.

27. The method of claim 23, wherein a dosage unit form of the polymer comprises 1 g to 6 g of the polymer.

28. A method of weight-loss or improving the body appearance in a healthy subject, comprising a step of orally administering to a subject the polymer according to claim 1.
